SEQUENCE LISTING (6 sequences)

SEQ ID NO: 1 - protein, 262 aa, Homo sapiens
MWFLVLCLALSLGGTGAAPPIQSRIVGGWECEQHSQPWQAALYHFSTFQCGGILVHRQWVLTAAHCISDNYQLWLGRHNLFDDENTAQFVHVSESFPHPGFNMSLLENHTRQADEDYSHDLMLLRLTEPADTITDAVKVVELPTEEPEVGSTCLASGWGSIEPENFSFPDDLQCVDLKILPNDECKKAHVQKVTDFMLCVGHLEGGKDTCVGDSGGPLMCDGVLQGVTSWGYVPCGTPNKPSVAVRVLSYVKWIEDTIAENS

SEQ ID NO: 2 - protein, 262 aa, Homo sapiens
MWFLVLCLALSLGGTGAAPPIQSRIVGGWECEQHSQPWQAALYHFSTFQCGGILVHRQWVLTAAHCISDNYQLWLGRHNLFDDENTAQFVHVSESFPHPGFNMSLLENHTRQADEDYSHDLMLLRLTEPADTITDAVKVVELPTQEPEVGSTCLASGWGSIEPENFSFPDDLQCVDLKILPNDECKKVHVQKVTDFMLCVGHLEGGKDTCVGDSGGPLMCDGVLQGVTSWGYVPCGTPNKPSVAVRVLSYVKWIEDTIAENS

SEQ ID NO: 3 - DNA, 19 bp, Artificial Sequence
  forward primer
tggcgtatgtgtggaaccg

SEQ ID NO: 4 - DNA, 20 bp, Artificial Sequence
  reverse primer
ctcgcagtagggaacagcaa

SEQ ID NO: 5 - DNA, 22 bp, Artificial Sequence
  forward primer
ctggatagcctttcttctgctg

SEQ ID NO: 6 - DNA, 20 bp, Artificial Sequence
  reverse primer
gcacactgtgtccgaactca

(19) United States
(12) Patent Application Publication
(10) Pub. No.: US 2014/0134152 A1
(43) Pub. Date: May 15, 2014

(54) COMPOSITIONS AND METHODS FOR TREATING DIABETES

(71) Applicants: Mark S Willimas, Plymouth, MN (US); Matthew L. Charles, St. Louis Park, MN (US)

(72) Inventors: Mark S Willimas, Plymouth, MN (US); Matthew L. Charles, St. Louis Park, MN (US)

(73) Assignee: DiaMedica Inc., Winnipeg (CA)

(21) Appl. No.: 14/116,115

(22) PCT Filed: May 4, 2012
(86) PCT No.: PCT/US2012/036556

§ 371 Date: Feb. 3, 2014

Related U.S. Application Data
(60) Provisional application No. 61/483,412, filed on May 6, 2011.
(60) Provisional application No. 61/523,010, filed on Aug. 12, 2011.
(60) Provisional application No. 61/565,926, filed on Dec. 1, 2011.
(60) Provisional application No. 61/566,481, filed on Dec. 2, 2011.

(51) Int. Cl. A61K 38/48

(52) U.S. Cl. CPC A61K 38/4853
USPC 424/94.64; 435/23; 435/226; 435/369; 435/358

(57) ABSTRACT

Embodiments of the present invention relate to compositions and methods of treating patients for type 1 diabetes mellitus (T1D) by administering a therapeutically effective dose of recombinant human KLK1, variants of KLK1, or active fragments thereof. Such patients may be increase risk patients for developing T1D or T1D patients in the Honeymoon Phase. Such treatment may be expected to prevent or delay the onset of T1D, to ameliorate the symptoms of T1D, or to ameliorate the extent to which the T1D manifests.

### CROSS-REFERENCE TO RELATED APPLICATIONS

This application claims the benefit under 35 U.S.C. §119(e) of U.S. Provisional Patent Application No. 61/483,412 filed May 6, 2011; U.S. Provisional Patent Application No. 61/523,010 filed Aug. 12, 2011; U.S. Provisional Patent Application No. 61/565,926 filed Dec. 1, 2011; and U.S. Provisional Patent Application No. 61/566,481 filed Dec. 2, 2011. The foregoing applications are incorporated herein by reference in their entirety.

### STATEMENT REGARDING THE SEQUENCE LISTING

The Sequence Listing associated with this application is provided in text format in lieu of a paper copy, and is hereby incorporated by reference into the specification. The name of the text file containing the Sequence Listing is DIAM_{—}001_{—}02WO_ST25.txt. The text file is about 6 KB, was created on May 4, 2012, and is being submitted electronically via EFS-web, concurrent with the filing of the specification.

### BACKGROUND

1. Technical Field

Embodiments of the present invention include recombinant forms of human tissue kallikrein-1 (KLK1), and related compositions and methods for treating various aspects of diabetes and delaying the onset of diabetes.

2. Description of the Related Art

Type 1 diabetes (T1D), also known as Insulin Dependent Diabetes Mellitus (IDDM), is a form of diabetes mellitus that results from autoimmune destruction of insulin-producing beta cells of the pancreas. The subsequent lack of insulin leads to increased blood and urine glucose. The classical symptoms are polyuria (frequent urination), polydipsia (increased thirst), polyphagia (increased hunger), and weight loss.

T1D is a chronic, eventually fatal, disease in which exogenous insulin administration is the only treatment available. However, insulin administration only treats the symptoms of T1D, and does treat the underlying disease. Injection is the most common method of administering insulin; insulin pumps and inhaled insulin have been available at various times. Pancreas and islet transplants have been used to treat T1D; however, islet transplants are currently still at the experimental trial stage and require donation of cadaver tissue and treatment with immunosuppressive drugs. Currently, T1D treatment with insulin must be continued indefinitely in all cases. Treatment is burdensome for many patients. Complications may be associated with both low blood sugar due to administration of too much insulin, and high blood sugar due to administration of insufficient amounts of insulin. Low blood sugar may lead to seizures or episodes of unconsciousness and requires emergency treatment. High blood sugar may lead to increased fatigue and can also result in long term damage to organs. Additionally, T1D diabetic patients are at much higher risk of developing cardiovascular disease, as well as retinopathy, neuropathy and nephropathy due to high blood sugar levels.

The pathophysiology in T1D is basically a destruction of beta cells in the pancreas due to an autoimmune reaction, regardless of which risk factors or causative entities have been present. Individual risk factors can have separate pathophysiological processes to, in turn, cause this beta cell destruction. Still, a process that appears to be common to most risk factors is an autoimmune response towards beta cells, involving an expansion of autoreactive CD4+ T helper cells and CD8+ T cytotoxic T cells, autoantibody-producing B cells and activation of the innate immune system.

T1D is not currently preventable. Therapies are emerging with the goal of preventing T1D at the latent autoimmune stage. However, such therapies have not proven to be effective long term in halting the autoimmune reaction that destroys insulin producing beta cells. Such therapies include:
Immunosuppressive drugs—Cyclosporine A, an immunosuppressive agent, has apparently halted destruction of beta cells (on the basis of reduced insulin usage), but its nephrotoxicity and other side effects make it highly inappropriate for long-term use
Anti-CD3 antibodies, including teplizumab and otelixizumab, have evidence of preserving insulin production in newly diagnosed type 1 diabetes patients. A probable mechanism of this effect is preservation of regulatory T cells that suppress activation of the immune system and thereby maintain immune system homeostasis and tolerance to self-antigens
An anti-CD20 antibody, rituximab, inhibits B cells and has been shown to provoke C-peptide responses three months after diagnosis of T1D, but long-term effects of this have not been reported.
GAD65 vaccine—Injections with a vaccine containing GAD65, an autoantigen involved in T1D, has in clinical trials delayed the destruction of beta cells when treated within six months of diagnosis. Patients treated with the substance showed higher levels of regulatory cytokines, thought to protect beta cells.

One objective of the instant invention is a treatment that will delay or prevent the onset of T1D in increased risk patients for development of the disease. Another objective of the invention is a treatment that will halt and reverse the autoimmune attack on beta cells in patients with T1D, including, for instance, those patients with recent onset T1D who are in the honeymoon phase of T1D, patients that have established T1D, and patients with latent autoimmune diabetes of adults (LADA).

### BRIEF SUMMARY OF THE INVENTION

The present invention includes methods of treating patients with type 1 diabetes (T1D) comprising administering a therapeutically effective dose of a human tissue kallikrein-1 (KLK1) polypeptide, variants of KLK1, or active fragments thereof to the patient. Certain embodiments include methods of treating a subject that does not have T1D, but is at increased risk for developing T1D, as characterized, for instance, by the presence or levels of one or more biomarkers. Other embodiments include methods of treating patients in the honeymoon phase of type 1 diabetes, including, for example, patients that have about 10-20% beta-cells (also referred to as recent onsent T1D), relative to a healthy patient or the same patient at an earlier time point, and that still produce insulin. Also included are methods of treating patients with established type 1 diabetes and patients with latent autoimmune diabetes of adults (LADA), for example, to halt the autoimmune reaction and reverse the diabetes. In some embodiments, the treatment comprises administering a therapeutically effective dose of recombinant human KLK1, variants of KLK1, or active fragments thereof to the patient.

Such treatment may be expected to delay the onset of type 1 diabetes, halt or reverse the progress of type 1 diabetes, or to ameliorate the extent to which the type 1 diabetes manifests.

Certain embodiments include methods for treating type 1 diabetes comprising administering a therapeutically effective amount of a KLK1 polypeptide to a patient. In some embodiments, the KLK1 polypeptide has serine protease activity and comprises an amino acid sequence having at least 95% sequence identity to SEQ ID NO:2, or residues 25-262 of SEQ ID NO:2. In certain embodiments, the KLK1 polypeptide retains an E145 substitution, an A188 substitution, or both, relative to SEQ ID NO:1. In specific embodiments, the KLK1 polypeptide retains an E145Q substitution, an A188V substitution, or both, relative to SEQ ID NO:1. In some embodiments, the KLK1 polypeptide comprises or consists of residues 25-262 SEQ ID NO:2.

In particular embodiments, the patient is at increased risk of developing type 1 diabetes, or has recent onsent or established type 1 diabetes, or LADA. In certain embodiments, the therapeutically effective amount of the KLK1 polypeptide provides an attenuation of autoimmune reaction against the pancreatic beta cells in the patient. In some embodiments, the patient is a mammal. In specific embodiments, the mammal is a human. In particular embodiments, the mammal is a domesticated animal, such as a cat or dog.

Some embodiments further comprise the step of measuring the level of a biomarker in the patient after treatment to assess the effectiveness of said treatment. In particular embodiments, the patient's serum C-peptide levels are increased compared to the serum C-peptide levels of the patient prior to onset of treatment with the KLK1. In some embodiments, the number or level of regulatory T cells (Tregs) or level of IDO expression is increased compared to levels in the patient prior to onset of treatment with KLK1. In certain methods, these and other biomarkers can be used to adjust the dosing amount and/or dosing frequency, to improve the treatment regime. For example, certain embodiments include maintaining or reducing the frequency and/or dosage of the KLK1 polypeptide upon increase in one or more of said biomarkers, optionally where said increase has been maintained for at least about 1, 2, 3, 4, 5, 6, or 7 weeks, or at least about 2, 3, or 4 months, or longer.

In some embodiments, the fasting blood glucose level of the patient is reduced compared to the fasting blood glucose level of the patient prior to onset of treatment with the KLK1. In some embodiments, the level of HbA1c of the patient is reduced compared to the HbA1c level of the patient prior to onset of treatment with the KLK1. In particular embodiments, the level of ketone bodies in the patient is reduced compared to the level of ketone bodies prior to onset of treatment with the KLK1. In certain methods, these and other biomarkers can be used to adjust the dosing amount and/or dosing frequency, to improve the treatment regime. For instance, certain embodiments include maintaining or reducing the frequency and/or dosage of the KLK1 polypeptide upon reduction of one or more of said biomarkers, optionally where said reduction has been maintained for at least about 1, 2, 3, 4, 5, 6, or 7 weeks, or at least about 2, 3, or 4 months, or longer.

Also included are methods for delaying onset of type 1 diabetes, and reversing recent onsent, LADA, and established type 1 diabetes in a patient, comprising administering a therapeutically effective amount of a KLK1 polypeptide (e.g., a polypeptide comprising the amino acid sequence of SEQ ID NO: 2, or residues 25-262 of SEQ ID NO:2).

In some embodiments, the patient does not have type 1 diabetes but is at increased risk for developing type 1 diabetes. In these and related embodiments, the patient may have one or more biomarkers associated with increased risk of T1D. Examples of such biomarkers include, without limitation, any one or more of certain HLA-DQB1 (IDDM1) alleles associated with T1D, increased antibodies against insulin, increased antibodies against islets, increased antibodies against glutamic acid decarboxylase (GAD), increased antibodies against IA2 (ICA512), increased circulating T cells that react with beta cell antigens, increased insulitis, increased inflammation of the pancreas, increased ketone bodies, decreased suppressor (regulatory) T cells (Tregs) (CD4+ cells that are also CD25+/Foxp3+), increased HbA1c levels, decreased C-peptide levels, and decreased IDO (Indoleamine-pyrrole 2,3-dioxygenase) levels, optionally relative to a healthy control or other reference standard.

Also included are methods of inhibiting a CD8+ cytotoxic T cell mediated autoimmune response against islets and/or beta cells (autoreactive CD8+ T cells), comprising administering a KLK1 polypeptide to a subject. Optionally the level or levels of autoreactive CD8+ T cells are measured prior to administration and following administration of a KLK1 polypeptide, wherein administration of a KLK1 polypeptide is continued until a decrease in the levels of autoreactive CD8+ T cells is observed. In some embodiments, the dosage levels and/or dosage frequency of KLK1 polypeptide administered to the patient is increased until a decrease in the levels of autoreactive CD8+ T cells is observed. Certain embodiments include maintaining or reducing the frequency and/or dosage of the KLK1 polypeptide upon decrease in the levels of autoreactive CD8+ T cells, optionally where said decrease has been maintained for at least about 1, 2, 3, 4, 5, 6, or 7 weeks, or 2, 3 or 4 months, or longer. In certain embodiments, the KLK1 polypeptide comprises the amino acid sequence of SEQ ID NO:2, or residues 25-262 of SEQ ID NO:2.

Certain methods further comprise determining circulating C-peptide levels in the patient optionally prior to administration and following administration of a KLK1 polypeptide, wherein administration of the KLK1 polypeptide is continued until an increase in circulating C-peptide levels is observed. Some methods further comprise determining indoleamine-pyrrole 2,3-dioxygenase (IDO) levels in the patient optionally prior to administration and following administration of a KLK1 polypeptide, wherein administration of a KLK1 polypeptide is continued until an increase in IDO levels is observed. Specific embodiments include measuring IDO mRNA levels in splenic dendritic cells (DCs) as a biomarker of treatment effectiveness.

Certain embodiments include methods for determining efficacy of administrating a KLK1 polypeptide to a patient with type 1 diabetes, the method comprising measuring the circulating C-peptide levels or IDO levels optionally prior to administration and following administration of a KLK1 polypeptide, wherein administration of a KLK1 polypeptide is continued until an increase in circulating C-peptide levels or IDO levels is observed. In some embodiments, the dosage levels and/or dosage frequency of KLK1 polypeptide administered to the patient is increased until an increase in C-peptide levels is observed. Certain embodiments include maintaining or reducing the frequency and/or dosage of the KLK1 polypeptide upon increase in C-peptide levels or IDO levels, optionally where said increase has been maintained for at least about 1, 2, 3, 4, 5, 6, or 7 weeks, or 2, 3 or 4 months, or longer.

Other embodiments include methods for determining efficacy of administrating a KLK1 polypeptide to a patient with type 1 diabetes, comprising measuring the regulatory T-cell levels, including CD4+ CD25+Foxp3+ cells, optionally prior to administration and following administration of a KLK1 polypeptide, wherein administration of a KLK1 polypeptide is continued until an increase in regulatory T-cell levels is observed. In some embodiments, the dosage levels and/or dosage frequency of KLK1 polypeptide administered to the patient is increased until an increase in regulatory T-cell levels is observed. Certain embodiments include maintaining or reducing the frequency and/or dosage of the KLK1 polypeptide upon increase in regulatory T-cell levels, optionally where said increase has been maintained for at least about 1, 2, 3, 4, 5, 6, or 7 weeks, or 2, 3 or 4 months, or longer.

Certain embodiments include methods for determining efficacy of administrating a KLK1 polypeptide to a patient with type 1 diabetes, the method comprising measuring an autoreactive beta cell antibody. Examples of autoreactive beta cell antibodies detected in human T1D patients include, without limitation, islet-cell antibodies (ICA), antibodies against insulin, antibodies against protein tyrosine phosphatase (IA2/ICA 512), and antibodies against glutamic acid decarboxylase (GAD). Optionally the level or levels of autoreactive beta cell antibody are measured prior to administration and following administration of a KLK1 polypeptide, wherein administration of a KLK1 polypeptide is continued until a decrease in an autoreactive beta cell antibody level is observed. In some embodiments, the dosage levels and/or dosage frequency of KLK1 polypeptide administered to the patient is increased until a decrease in an at least one autoreactive beta cell antibody level is observed. Certain embodiments include maintaining or reducing the frequency and/or dosage of the KLK1 polypeptide upon decrease in an at least one autoreactive beta cell antibody level, optionally where said decrease has been maintained for at least about 1, 2, 3, 4, 5, 6, or 7 weeks, or 2, 3 or 4 months, or longer.

Particular embodiments include methods for determining efficacy of administrating a KLK1 polypeptide to a patient with type 1 diabetes, comprising measuring indoleamine-pyrrole 2,3-dioxygenase (IDO) levels optionally prior to administration and following administration of a KLK1 polypeptide, wherein administration of a KLK1 polypeptide is continued until an increase in IDO levels is observed. Certain embodiments include measuring IDO mRNA levels in splenic dendritic cells (DCs). In some embodiments, the dosage levels and/or dosage frequency of KLK1 polypeptide administered to the patient is increased until an increase in IDO levels is observed.

In certain methods, the KLK1 polypeptide is administered by intraperitoneal or subcutaneous injection.

Certain embodiments relate to isolated, recombinant KLK1 polypeptides, comprising the amino acid sequence of SEQ ID NO:2, residues 19-262 of SEQ ID NO:2, residues 25-262 of SEQ ID NO:2, or a variant having an amino acid sequence at least 95% identical thereto, where the variant retains an E145 substitution, an A188 substitution, or both, relative to SEQ ID NO:1. In specific embodiments, the isolated KLK1 variant retains an E145Q substitution, an A188V substitution, or both, relative to SEQ ID NO:1. In some embodiments, the isolated KLK1 polypeptide comprises residues 25-262 of SEQ ID NO:2. In some embodiments, the KLK1 polypeptide further comprises a heterologous fusion partner.

Also included are isolated polynucleotides, which encode the KLK polypeptides described herein, such as SEQ ID NO:2, residues 19-262 of SEQ ID NO:2, residues 25-262 of SEQ ID NO:2, and variants thereof. Certain embodiments include vectors, where the polynucleotide is (optionally) operably linked to one or more regulatory elements, for example, promoters, enhancers, etc. Also included are host cells, comprising one or more of the polynucleotides/vectors described herein, and/or recombinant forms of the KLK1 polypeptides described herein. In certain embodiments, the host cell is 293 cell or a CHO cell.

Certain embodiments include pharmaceutical compositions, comprising one or more of a) a KLK polypeptide described herein (e.g., residues 25-262 of SEQ ID NO:2), b) a polynucleotide described herein, or c) a vector described herein, and a physiologically acceptable carrier. In certain embodiments, the pharmaceutical compositions are for the treatment of established type 1 diabetes (T1D), honeymoon phase T1D or recent onset T1D, LADA including recent onset LADA and established LADA, and/or gestational diabetes, among other conditions described herein. In some embodiments, the pharmaceutical compositions are for the treatment of pre-diabetes, i.e., for the treatment of a patient that does not have type 1 diabetes (T1D) but is at risk for developing T1D, as described herein.

### BRIEF DESCRIPTION OF THE DRAWINGS

FIG. 1A is an SDS-PAGE gel stained with Coomassie Blue stain of various amounts of recombinant human KLK1 purified from CHO or 293 cell lines following transient transfection. Lane 1 is a pre-stained protein ladder, the molecular weights of the standards are written on the side (in kDa). Lanes 2-5 have KLK1 purified from CHO cells (lane 2, 14 μg protein; lane 3, 7 μg protein; lane 4, 3.5 μg protein; lane 5, 1.35 μg protein). Lane 6 has 14 μl of KLK1 protein purified from transient transfection of 293 cells.

FIG. 1B is a Western blot stained with mouse anti-human KLK1 polyclonal antibodies of various amounts of recombinant human KLK1 purified from CHO or 293 cell lines following transient transfection. Lanes 1 and 6 are loaded with a pre-stained protein ladder, the molecular weights of the standards are written on the side (in kDa). Lanes 2-5 have KLK1 purified from CHO cells (lane 2, 5 μl protein; lane 3, 2.5 μl protein; lane 4, 1.25 μl protein). Lane 5 has 2.5 μl of KLK1 protein purified from transient transfection of 293 cells.

FIG. 2 is a graph of the Kaplan-Meier curve for induction of diabetes in female NOD mice treated with recombinant human KLK1 at the following doses: Group 1, negative control, vehicle only “vehicle”); Group 2, 0.08 Units KLK1 per mouse daily (“low daily”); Group 3, 0.4 Units KLK1 per mouse daily (“medium daily”); Group 4, 2 Units KLK1 per mouse daily (“high daily”); Group 5, 2 Units KLK1 per mouse every 3 days (“high 3×/wk”); Group 6, 2 Units KLK1 per mouse every 7 days (“high 1×/wk”).

FIGS. 3A-3F depict the fasting blood glucose levels of individual NOD mice treated with recombinant human KLK1. FIG. 3A (Group 1, vehicle); FIG. 3B (Group 2, low daily); FIG. 3C (Group 3, medium daily); FIG. 3D (Group 4, high daily); FIG. 3E (Group 5, high 3×/wk); FIG. 3F (Group 6, high 1×/wk). The x-axis depicts the weeks of treatment with recombinant human KLK1, and the y-axis depicts the blood glucose levels (mg/dl). Each line represents the blood glucose level of an individual NOD mouse. The boxes depict when the NOD mouse became positive for diabetes.

FIGS. 4A-4F show the blood glucose levels in individual mice treated with recombinant human KLK1 following an intraperitoneal glucose tolerance tests (GTT). Each of the figures shows a graph of GTT performed on animals when they were 19 weeks old (day 96 of KLK1 treatment), 21 weeks old (day 109 of KLK1 treatment), and 23 weeks old (day 123 of KLK1 treatment). FIG. 4A (Group 1, vehicle); FIG. 4B (Group 2, low daily); FIG. 4C (Group 3, medium daily); FIG. 4D (Group 4, high daily); FIG. 4E (Group 5, high 3×/wk); FIG. 4F (Group 6, high 1×/wk). The x-axis depicts the time in minutes after glucose injection, and the y-axis depicts the blood glucose levels (mg/dl). Each line represents the blood glucose level of an individual NOD mouse.

FIG. 5 is a graph of the average systolic blood pressure in mice treated with recombinant human KLK1 at treatment day 77 (left graph), treatment day 98 (middle graph), and treatment day 126 (right graph). Blood pressure and heart rate measurements were taken from 5 random mice, using blood pressure monitoring system (IITC Life Science Inc.), which obtains and records systolic, diastolic, and mean pressure and heart rate utilizing photoelectric sensor detection of blood pressure pulses.

FIG. 6 is a graph of the results of FACS analysis of the percent of CD25+ Foxp3+ cells compared to the total CD4+ T cells isolated from spleens of animals treated with recombinant human KLK1 for 18 weeks. Treatment groups are as follows: Group 1, vehicle only; Group 2, low daily; Group 3, medium daily; Group 4, high daily; Group 5, high 3×/wk; Group 6, high 1×/wk.

FIG. 7 is a graph of the effects of 4 weeks of KLK1 treatment on beta cells in NOD mice. Pancreata from NOD mice were isolated and stained for EdU (to detect replicating cells) and insulin (to detect beta cells). At least three slides per mouse, 150 micrometres apart per slide were analyzed. The total number of double positive cells for EdU and insulin was divided by the total number of insulin positive (beta cells) and multiplied by 100. Group 1, vehicle only; Group 2, low daily; Group 3, medium daily; Group 4, high daily.

FIG. 8 is a graph of average insulitis grades observed histologically in islets after 4 weeks of KLK1 treatment in NOD mice. Pancreatic sections were analyzed under fluorescent microscopy for islet infiltration based on the insulitis scale of 0 to 4 described herein. The graph represents the average insulitis score in arbitrary units+/−SEM. Group 1, vehicle only; Group 2, low daily; Group 3, medium daily; Group 4, high daily.

FIG. 9 is a graph of the average insulitis grades observed histologically in islets after 18 weeks of KLK1 treatment in NOD mice. Pancreatic sections were analyzed under fluorescent microscopy for islet infiltration based on the insulitis scale of 0 to 4 described herein. The graph represents the average insulitis score in arbitrary units+/−SEM. Minimum of 80 islets per mouse were examined. n—number of animals in each group (color-coded).

FIG. 10 is a graph of the average beta cell mass after 18 weeks of KLK1 treatment in NOD mice. Pancreatic sections were fluorescently stained for insulin; and beta cell masses were calculated by measuring insulin-positive stained area of each islet on the section, calculating summarized insulin-positive stained area for each section, which was then divided by the total pancreas area of the section, and resultant value multiplied by the total pancreas weight. Three slides per mouse were analyzed in blinded fashion with the sections being 150 microns apart. n—number of animals in each group (color-coded).

FIG. 11A is a graph of the composition of islet infiltration after 18 weeks of KLK1 treatment. The data is expressed as an average ratio of CD4 cells to CD8 cells for indicated treatment group±S.E.M. n—number of animals analyzed in each group.

FIG. 11B depicts the frequency of CD8+ T cells in lymphocytes' gates (%)±SEM (three graphs on left side) and T-regulatory cells population out of total CD4+ T cells (%)±SEM (three graphs on right side) in the peripheral blood (top two graphs), spleen (middle two graphs) and pancreatic lymph nodes (bottom two graphs). Single-cell suspensions obtained from spleens, pancreatic lymph nodes, and peripheral blood samples of all animals remaining non-diabetic by the end of KLK1 treatment were stained with fluorescently-labeled antibodies for CD4, CD25, Foxp3, and CD8 markers and subjected to FACS analysis. Percentages of CD8-positive T cells within lymphocytes' gates; and T-regulatory cells (triple-positive for CD4, CD25 and Foxp3 markers) within total CD4 population were calculated, and data expressed as an average percentage for each experimental group±SEM. n—number of animals analyzed in each group. p<0.05 vs. Group 1 via 1-way Anova analysis with post-hoc Turkey multiple comparisons test

FIG. 12 is a graph of the serum insulin levels during KLK1 treatment. Sera from non-fasting experimental mice was drawn and insulin levels measured using a mouse insulin ELISA (Millipore) according to the manufacturers' protocol. The x-axis depicts the days of treatment with recombinant human KLK1, and the y-axis depicts the insulin concentration (ng/ml). Data are expressed as an average insulin concentration for each experimental group+/−SEM.

FIG. 13 is a graph of the serum TGF-β concentrations during KLK1 treatments. Sera from non-fasting experimental mice was isolated and TGF-β concentrations were measured by ELISA using a mouse TGF-β kit (R&D) according to the manufacturers' protocol. The x-axis depicts the days of treatment with recombinant human KLK1, and the y-axis depicts the TGF-β concentration (ng/ml). Data are expressed as an average TGF-β concentration for each experimental group+/−SEM.

FIG. 14 is a graph of the serum mouse C-peptide levels during KLK1 treatment. Sera from non-fasting experimental mice was drawn and mouse C-peptide levels measured using a mouse C-peptide ELISA (ALPCO) according to the manufacturers' protocol. The x-axis depicts the days of treatment with recombinant human KLK1, and the y-axis depicts the serum C-peptide concentration (nM). Data are expressed as an average C-peptide concentration for each experimental group+/−SEM.

FIG. 15 is a graph of the serum mouse C-peptide levels during a shortened KLK1 treatment regimen (10 weeks). Sera were drawn from mice and mouse C-peptide levels measured as described in FIG. 14. The x-axis depicts the weeks of treatment with recombinant human KLK1, and the y-axis depicts the C-peptide concentration (nM). Data are expressed as an average C-peptide concentration for each experimental group+/−SEM.

FIG. 16 is a graph that depicts IDO mRNA levels in total splenocytes and splenocytes DCs from NOD mice treated with KLK1. Data are expressed as a normalized average of IDO mRNA ΔCt values relative to the ΔCt values of an endogenous reference gene (beta-actin)+/−SEM.

### DETAILED DESCRIPTION

Tissue kallikreins are members of a gene super family of serine proteases comprising at least 15 separate and distinct proteins (named tissue kallikrein 1 through 15) that share similar gene and protein sequence (Yousef et al., Endocrine Rev. 22: 184-204, 2001). In humans and animal tissues, tissue kallikrein 1 (KLK1 or pancreatic/renal kallikrein) is the member known to cleave kininogen into lysyl-bradykinin (kallidin). Bradykinin is a peptide that causes blood vessels to dilate (enlarge), and therefore causes blood pressure to lower. Kallidin is identical to bradykinin with an additional lysine residue added at the N-terminal end and signals through the bradykinin receptor. KLK1 may be a ubiquitous or multiple function enzyme, in addition to its recognized role in hypertension regulation. As used herein, the term “human tissue kallikrein” and KLK1 are synonymous.

A “variant” or “mutant” of a starting or reference polypeptide is a polypeptide that 1) has an amino acid sequence different from that of the starting or reference polypeptide and 2) was derived from the starting or reference polypeptide through either natural or artificial (manmade) mutagenesis. Such variants include, for example, deletions from, and/or insertions into and/or substitutions of, residues within the amino acid sequence of the polypeptide of interest. A variant amino acid, in this context, refers to an amino acid different from the amino acid at the corresponding position in a starting or reference polypeptide sequence. Any combination of deletion, insertion, and substitution may be made to arrive at the final variant or mutant construct, provided that the final construct possesses the desired functional characteristics. The amino acid changes also may alter post-translational processes of the polypeptide, such as changing the number or position of glycosylation sites. A polypeptide or polynucleotide variant generally has at least about 80% 85%, 90%, 91%, 92%, 93%, 94%, 95%, 96%, 97%, 98%, 98.5%, 99%, or 99.5% amino acid or nucleotide sequence identity with a reference sequence. It should be noted, however, that to qualify as a “variant” such polypeptide or polynucleotide sequence must differ from a reference sequence in at least one amino acid residue or nucleotide (which may be replaced or omitted). Variants may also include sequences added to the reference polypeptide to facilitate purification, to improve metabolic half-life or to make the polypeptide easier to identify, for example, a His tag or a pegylation sequence.

A “wild type” or “reference” sequence or the sequence of a “wild type” or “reference” protein/polypeptide may be the reference sequence from which variant polypeptides are derived through the introduction of mutations. In general, the “wild type” amino acid sequence for a given protein is the sequence that is most common in nature. Similarly, a “wild type” gene sequence is the polynucleotide sequence for that gene which is most commonly found in nature. Mutations may be introduced into a “wild type” gene (and thus the protein it encodes) either through natural processes or through human induced means. The products of such processes are “variant” or “mutant” forms of the original “wild type” protein or gene.

“Percent (%) amino acid sequence identity” with respect to a polypeptide is defined as the percentage of amino acid residues in a candidate sequence that are identical with the amino acid residues in the reference sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or Megalign (DNASTAR) software. Those skilled in the art can determine appropriate parameters for measuring alignment, including any algorithms needed to achieve maximal alignment over the full length of the sequences being compared. The ALIGN-2 program is publicly available through Genentech, Inc., South San Francisco, Calif.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y,

where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

“Percent (%) nucleic acid sequence identity” is defined as the percentage of nucleotides in a candidate sequence that are identical with the nucleotides in a reference polypeptide-encoding nucleic acid sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

### Compositions, Polypeptides, Nucleic Acids

The disclosure provides for compositions including a KLK1 polypeptide as well as nucleic acids encoding a KLK1 polypeptide. In particular embodiments, the KLK1 polypeptide is a recombinant human polypeptide. Recombinant human KLK1 (rhKLK1) can provide certain advantages over other sources of KLK1, such as porcine KLK1 (e.g., porcine KLK1 isolated from pancreas). For example, rhKLK1 is less immunogenic in human patients compared to porcine KLK1, partly because the sequence of porcine KLK1 differs in amino acid sequence compared to human KLK1 (i.e., porcine KLK1 has 67% amino acid homology to human KLK1). Reduced immunogenicity can be advantageous, for instance, where a treatment protocol requires KLK1 administration over a prolonged period of time, such as 1, 2, 3, 4, 5, 6 or 7 weeks or months or more. Also, rhKLK1 used as a therapeutic in humans has a higher specificity to human substrate (kininogen) than the specificity of porcine KLK1 to human kininogen, and may thus have higher biological actitivy. Further, the recombinant system used to manufacture rhKLK1 can be designed to use chemically defined, animal-component free media that has far less risk of contamination with pathogens such as viruses, prions and other materials relative to porcine KLK1 sourced from pig pancreas. rhKLK1 can also be manufactured to higher purity with lower batch-to-batch variability and lower host cell protein contamination than porcine KLK1 isolated from pig pancreas.

An embodiment of the present invention can be human tissue kallikrein precursor polypeptide (kidney/pancreas/salivary gland kallikrein) (KLK1) and has the following sequence (SEQ ID NO: 1):

NP_002248.1 GI:4504875 Homo sapiens KLK1 human
1-18 signal peptide
19-24 propeptide
25-262 mature peptide
>gi|4504875|ref|NP_002248.1|kallikrein-1 preproprotein
[Homo sapiens]
(SEQ ID NO: 1)
MWFLVLCLALSLGGTGAAPPIQSRIVGGWECEQHSQPWQAALYHFSTFQC 50
GGILVHRQWVLTAAHCISDNYQLWLGRHNLFDDENTAQFVHVSESFPHPG 100
FNMSLLENHTRQADEDYSHDLMLLRLTEPADTITDAVKVVELPTEEPEVG 150
STCLASGWGSIEPENFSFPDDLQCVDLKILPNDECKKAHVQKVTDFMLCV 200
GHLEGGKDTCVGDSGGPLMCDGVLQGVTSWGYVPCGTPNKPSVAVRVLSY 250
VKWIEDTIAENS

When comparing the above Genbank amino acid sequence for human KLK1 to the amino acid sequence of a cDNA for human KLK1 purchased from Origene (Rockville, Md., USA), two apparent amino acid substitutions were detected that may result from single-nucleotide polymorphism or SNP's between individuals within a species. The SNP's result in an apparent E to Q at amino acid residue 145 of 262, and an apparent A to V position 188 of 262, as depicted in SEQ ID NO:2. Among other potential advantages, recombinant human KLK1 with the two SNPs (SEQ ID NO: 2) appears to have higher expression levels in recombinant cell culture than the allele with the sequence identified as SEQ ID NO: 1. The two SNPs of SEQ ID NO:2 also appear to be more prevalent in the human population and thus a rhKLK1 therapeutic encoding one or both of the SNPs may be less immunogenic in humans, which can be advantageous if the rhKLK1 is administered daily for a period of several weeks or months. Naturally occurring genetic variations in the human KLK1 gene and protein (e.g., alleles of KLK1) have been described in human populations, and these KLK1 alleles have been associated with conditions such as hypertension and renal calcium clearance in humans (see Zhao et al., J Hypertens. 25:1821-7, 2007).

The inventors proceeded with expressing, purifying and testing the KLK1 polypeptide described in SEQ ID NO:2. Certain embodiments therefore include polypeptides that comprise the amino acid sequence of SEQ ID NO:2, and variants thereof (e.g., 90% identity, 95% identity) that optionally retain one or both of the substitution noted above (e.g., an E145 substitution, an A188 substitution), relative to SEQ ID NO:1. Also included are pharmaceutical compositions that comprise the polypeptide of SEQ ID NO:2, residues 19-24 of SEQ ID NO:2, or residues 25-262 of SEQ ID NO:2, and related methods of treating T1D.

(SEQ ID NO: 2):
1-18 signal peptide
19-24 propeptide
25-262 mature peptide
MWFLVLCLALSLGGTGAAPPIQSRIVGGWECEQHSQPWQAALYHFSTFQC 50
GGILVHRQWVLTAAHCISDNYQLWLGRHNLFDDENTAQFVHVSESFPHPG 100
FNMSLLENHTRQADEDYSHDLMLLRLTEPADTITDAVKVVELPTQEPEVG 150
STCLASGWGSIEPENFSFPDDLQCVDLKILPNDECKKVHVQKVTDFMLCV 200
GHLEGGKDTCVGDSGGPLMCDGVLQGVTSWGYVPCGTPNKPSVAVRVLSY 250
VKWIEDTIAENS

A gene coding for a human tissue kallikrein polypeptide of the present invention encodes a 262-amino acid tissue kallikrein polypeptide: a presumptive 17-amino acid signal peptide, a 7-amino acid proenzyme fragment and a 238-amino acid mature KLK1 protein.

In certain embodiments, a KLK1 polypeptide comprises a full length polypeptide, a propeptide (i.e., with the signal sequence removed) or a mature peptide (lacking signal peptide and pro sequence).

The term “active fragment” refers to smaller portions of a KLK1 polypeptide that retains the activity of a KLK1 polypeptide. In some embodiments, an active fragment retains serine protease activity of a KLK1 polypeptide that releases kallidin from a higher molecular weight precursor such as kininogen, or that cleaves a substrate similar to kininogen such as D-val-leu-arg-7 amido-4-trifluoromethylcoumarin described herein to release a colorimetric or fluorometric fragment. In some aspects, the KLK1 polypeptide or active fragment may directly bind a receptor to elicit the immunomodulatory effects that attenuate the autoimmune reaction that results in the anti-diabetic activity.

The disclosure also provides variants of a KLK1 polypeptide. In certain embodiments a variant retains serine protease activity and/or anti-diabetic activity. In some embodiments a KLK1 polypeptide has the percent amino acid sequence identity with a reference sequence described above, such as the full length, propeptide or mature KLK1 having a sequence of SEQ ID NO:2.

The present invention also contemplates the use of KLK1 fusion proteins. As used herein, KLK1 “fusion protein” includes a KLK1 polypeptide or polypeptide fragment linked to either another KLK1-polypeptide (e.g., to create multiple fragments), to a non-KLK1 polypeptide, or to both. A “non-KLK1 polypeptide” refers to a “heterologous polypeptide” having an amino acid sequence corresponding to a protein which is different from KLK1 protein, and which is derived from the same or a different organism. The KLK1 polypeptide of the fusion protein can correspond to all or a portion of a biologically active KLK1 amino acid sequence, for example, a fragment having serine protease activity and/or anti-diabetic activity. In certain embodiments, a KLK1 fusion protein includes at least one (or two) biologically active portion of a KLK1 protein. The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order.

The fusion partner may be designed and included for essentially any desired purpose provided they do not adversely affect the therapeutic activity of the KLK1 polypeptide. For example, in one embodiment, a fusion partner may comprise a sequence that assists in expressing the KLK1 protein (an expression enhancer) at higher yields than the native recombinant protein. Other fusion partners may be selected so as to increase the solubility of the KLK1 protein or to enable the protein to be targeted to desired tissues.

The disclosure provides nucleic acids encoding a KLK1 polypeptide. In certain embodiments and according to the present invention, DNA sequences encoding a human KLK1 polypeptide have been isolated and characterized. Further, human DNA may be utilized in eukaryotic and prokaryotic expression systems to provide isolatable quantities of KLK1 protein having biological and immunological properties of naturally-occurring KLK1 as well as in vivo and in vitro biological activities, in particular therapeutic activity or serine protease activity, of naturally-occurring KLK1. In some embodiments, an isolated nucleic acid codes for a KLK1 polypeptide that has serine protease activity and has at least about 80% 85%, 90%, 91%, 92%, 93%, 94%, 95%, 96%, 97%, 98%, 98.5%, 99%, or 99.5% amino acid sequence identity with a reference sequence, such as the full length, propeptide or mature KLK1 having a sequence of SEQ ID NO:2, or which optionally retains a SNP that encodes an E145 substitution, an A188 substitution, or both, relative to SEQ ID NO:1.

Illustrative of the present invention are cloned DNA sequences of human species origins and polypeptides suitably deduced therefrom which represent, respectively, the primary structural conformation of KLK1 of human species origins.

The KLK1 polypeptides described herein may be prepared by any suitable procedure known to those of skill in the art, such as by recombinant techniques. As one general example, KLK1 polypeptides may be prepared by a procedure including one or more of the steps of: (a) preparing a construct comprising a polynucleotide sequence that encodes a KLK1 polypeptide and that is operably linked to a regulatory element; (b) introducing the construct into a host cell; (c) culturing the host cell to express the KLK1 polypeptide; and (d) isolating the KLK1 polypeptide from the host cell. The construct and expression system may be such that the mature or active KLK1 is expressed from the host cell. Alternatively, the KLK1 polypeptide may be expressed in an inactive form, such as a propeptide, and the KLK1 serine protease activity may be activated (for example, by removing the “pro” sequence) after the KLK1 polypeptide is isolated form the host cell.

In order to express a desired polypeptide, a nucleotide sequence encoding the polypeptide, or a functional equivalent, may be inserted into appropriate expression vector, i.e., a vector which contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook et al., Molecular Cloning, A Laboratory Manual (1989), and Ausubel et al., Current Protocols in Molecular Biology (1989).

A variety of expression vector/host systems are known and may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems, including mammalian cell systems.

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan & Shenk, PNAS USA. 81:3655-3659, 1984). In addition, transcription enhancers, such as the Rous sarcoma virus (RSV) enhancer, may be used to increase expression in mammalian host cells.

Examples of useful mammalian host cell lines include monkey kidney CV1 line transformed by SV40 (COS-7, ATCC CRL 1651); human embryonic kidney line (293 or 293 cells sub-cloned for growth in suspension culture, Graham et al., J. Gen Virol. 36:59 (1977)); baby hamster kidney cells (BHK, ATCC CCL 10); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2). Other useful mammalian host cell lines include Chinese hamster ovary (CHO) cells, including DHFR-CHO cells (Urlaub et al., PNAS USA. 77:4216, 1980); and myeloma cell lines such as NSO and Sp2/0.

Prokaryotic or eukaryotic host expression (e.g., by bacterial, yeast and mammalian cells in culture) of exogenous DNA of the present invention obtained by genomic or cDNA cloning or by gene synthesis yields recombinant human KLK1 polypeptides described herein. KLK1 polypeptide products of cell culture expression in vertebrate (e.g., mammalian and avian) cells may be further characterized by freedom from association with human proteins or other contaminants, which may be associated with KLK1 in its natural mammalian cellular environment or in extracellular fluids such as plasma or urine. Products of typical yeast (e.g., Saccharomyces cerevisiae) or prokaryote (e.g., E. coli) host cells are free of association with any mammalian proteins. Depending upon the host employed, polypeptides of the invention may be glycosylated with mammalian or other eukaryotic carbohydrates. Polypeptides of the invention may also include an initial methionine amino acid residue (at position-1). Certain embodiments therefore include host cells (e.g., eukaryotic host cells such as CHO cells, 293 cells, etc.) that comprise a recombinant or introduced polynucleotide that encodes a KLK1 polypeptide described herein, such as the polypeptide of SEQ ID NO:1 or 2. Also included are host cells that comprise an (exogenous) polynucleotide that encodes a recombinant (e.g., non-naturally occurring) KLK-1 polypeptide described herein, such as the polypeptide of SEQ ID NO:1 or 2.

The cell culture expressed KLK1 polypeptides of the present invention may be isolated and purified by using, e.g., chromatographic separations or immunological separations involving monoclonal and/or polyclonal antibody preparations, or using inhibitors or substrates of serine proteases for affinity chromatography. As will be evident to those skilled in the art, SEQ ID NO 1 and SEQ ID NO 2 list the sequence for pre-pro KLK1. If the gene coding for either of these sequences is expressed in mammalian cells, the 17-amino acid signal peptide (residues 1-18) should result in the KLK1 polypeptide to be secreted by the cell, and the signal peptide removed by the cell. If it is desired to not have the polypeptide secreted, or if non-mammalian cells are used for expression, a gene encoding KLK1 may be generated in which the signal sequence is omitted or replaced with another sequence. The 7 amino acid pro-sequence (residues 19-24) will inhibit the serine protease activity of the KLK1 and should be removed to allow activity of the mature KLK1 polypeptide. The pro-sequence may be removed after the KLK1 polypeptide is isolated, for example by exposing the pro-KLK1 to trypsin under conditions that will allow cleavage of the pro-sequence, or by generating a gene encoding KLK1 in which the pro-sequence omitted or replaced with another sequence. Polypeptide products of the invention may be “labeled” by covalent association with a detectable marker substance (e.g., radiolabels such as I¹²⁵ and P³², nonisotopic labels such as avidin-biotin) to provide reagents useful in detection and quantification of KLK1 in solid tissue and fluid samples such as blood or urine. DNA products of the invention may also be labeled with detectable markers (for example, radiolabels such as I¹²⁵ or P³² and nonisotopic labels such as biotin) and employed in DNA hybridization processes to locate the KLK1 gene position and/or the position of any related gene family in a human, monkey and other mammalian species chromosomal map. The labeled DNA may also be used for identifying the KLK1 gene disorders at the DNA level and used as gene markers for identifying neighboring genes and their disorders.

In addition to recombinant production methods, KLK1 polypeptides may be produced by direct peptide synthesis using solid-phase techniques (see Merrifield, J. Am. Chem. Soc. 85:2149-2154, 1963). Protein synthesis may be performed using manual techniques or by automation. Automated synthesis may be achieved, for example, using Applied Biosystems 431A Peptide Synthesizer (Perkin Elmer). Alternatively, various fragments may be chemically synthesized separately and combined using chemical methods to produce the desired polypeptide. Also included is cell-free expression of proteins. These and related embodiments typically utilize purified RNA polymerase, ribosomes, tRNA and ribonucleotides; these reagents may be produced by extraction from cells or from a cell-based expression system.

KLK1 polypeptide products provided by the invention are products having a primary structural conformation of a naturally-occurring tissue kallikrein-1 to allow possession of one or more of the biological properties thereof and having an average carbohydrate composition which may differ from that of naturally-occurring tissue kallikrein-1.

KLK1 is a serine protease which cleaves low-molecular-weight kininogen resulting in the release of kallidin (lys-bradykinin). This activity of KLK1 may be measured in an enzyme activity assay by measuring either the cleavage of low-molecular-weight kininogen, or the generation of lys-bradykinin. Assays include examples wherein a labeled substrate is reacted with KLK1, and the release of a labeled fragment may be detected. One example of such a fluorogenic substrate suitable for KLK1 measurement of activity is D-val-leu-arg-7 amido-4-trifluoromethylcoumarin (D-VLR-AFC, FW 597.6) (Sigma, Cat #V2888 or Ana Spec Inc Cat #24137.) When D-VLR-AFC is hydrolyzed, the free AFC produced in the reaction can be quantified, for example, by fluorometric detection (excitation 400 nm, emission 505 nm according to the catalogue, but alternate excitation and emissions are possible, including excitation 360 nm, emission 460 nm) or by spectrophotometric detection at 380 nm (extinction coefficient=12,600 at pH 7.2). Other methods and substrates may also be used to measure KLK1 proteolytic activity.

KLK1 activity, measured in Units, Units/mg, or Units/ml, may be determined by comparing the relative activity of a KLK1 sample to the Kininogenase, Porcine standard acquired from the National Institute for Biological Standards and Control (NIBSC Product No. 78/543). For this standard, the assigned potency is 22.5 international units (IU) per 20 μg ampoule of porcine pancreatic kininogenase. Typically, serial dilutions are made of the standard, and the activity in an unknown sample of KLK1 is compared to the standard.

In embodiments, a composition (e.g., pharmaceutical composition) comprises a KLK1 polypeptide in combination with a physiologically acceptable carrier. Such carriers include pharmaceutically acceptable carriers, excipients, or stabilizers which are nontoxic to the cell or mammal being exposed thereto at the dosages and concentrations employed. Methods of formulation are well known in the art and are disclosed, for example, in Remington: The Science and Practice of Pharmacy, Mack Publishing Company, Easton, Pa., 19th Edition (1995).

The phrase “physiologically-acceptable” or “pharmaceutically-acceptable” refers to molecular entities and compositions that do not produce an allergic or similar untoward reaction when administered to a human. The preparation of an aqueous composition that contains a KLK1 protein as an active ingredient is well understood in the art. Typically, such compositions are prepared as injectables, either as liquid solutions or suspensions; solid forms suitable for solution in, or suspension in, liquid prior to injection can also be prepared. The preparations can also be emulsified.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

In some embodiments, a composition comprises a therapeutically effective amount of a KLK1 polypeptide. A therapeutically effective amount can be determined using standard dosage methods. In particular embodiments, a therapeutically effective amount includes an amount that lowers fasting glucose, that increases glucose tolerance, and/or decreases an autoimmune reaction against pancreatic beta cells.

Certain treatment methods described herein may also include steps of monitoring various biomarkers or other indicators of treatment, for instance, to evaluate the effectiveness of the treatment(s). In some embodiments, based on the results of such monitoring, a treatment schedule can be adjusted, for instance, by increasing the frequency and/or dosage levels of the KLK1 polypeptide(s). As one example of a biomarker, indoleamine-pyrrole 2,3-dioxygenase (IDO or INDO EC 1.13.11.52) “IDO” is an immunomodulatory enzyme produced by some alternatively activated macrophages and other immunoregulatory cells. In humans, IDO is encoded by the INDO gene (see Dai et al., Biochem. Biophys. Res. Commun. 168:1-8, 1990). Experiments described herein show that KLK1 treatment in mice is associated with increased expression of IDO in splenic dendritic cells (DC's), as measured by mRNA levels relative to an endogenous control (e.g., beta-actin).

Increases in IDO expression (e.g., as measured by mRNA or protein levels) could thus be used a biomarker to determine the effectiveness of KLK1 dose or treatment in patients. As one example, if KLK1 administration at a certain dose or treatment regimen results in increased IDO levels, for instance, increased mRNA levels in splenic DCs optionally relative to an endogenous control (e.g., an increase that is statistically significant), then that dose or treatment regimen may be viewed as being beneficial to patient. Alternatively, if the KLK1 dose or treatment regimen results in little or no increase in IDO levels (e.g., an increase that is non-statistically significant), then the T1D may be progressing in the patient, and the dosage amount and/or dosage frequency may be increased until an increase in IDO levels is observed. IDO mRNA levels can be measured, for example, by quantitative PCR (qPCR) methods known in the art. IDO protein levels can be measured, for example, by ELISA, or other methods known in the art. In specific embodiments, IDO levels are measured in splenic DCs, which can be isolated according to routine techniques in the art (see Example 4).

As another example of a biomarker, proinsulin C-peptide is a by-product of the insulin biosynthesis. It serves as a linker between the A- and the B-chains of insulin and facilitates the efficient assembly, folding, and processing of insulin in the endoplasmic reticulum. Equimolar amounts of C-peptide and insulin are then stored in secretory granules of the pancreatic beta cells and both are eventually released to the portal circulation. Newly diagnosed diabetes patients can have their C-peptide levels measured as a means of distinguishing type 1 diabetes and type 2 diabetes. C-peptide levels are measured instead of insulin levels because insulin concentration may be very low in the peripheral circulation and varies with the nutritional state. Patients with type 1 diabetes are unable to produce insulin, and will usually have decreased C-peptide levels. In contrast, C-peptide levels in type 2 patients are normal or higher than normal. Patients may also be injected with synthetic insulin prior to testing for C-peptide to help determine how much insulin these patients are still producing, or if they produce any at all. Measuring C-peptide is typically done via an ELISA assay on serum or blood plasma isolated from the patient. C-peptide has been found to be a bioactive peptide in its own right, with effects on microvascular blood flow and tissue health. C-peptide levels may be a general indicator of the health of beta cells in type 1 diabetic patients, and an indicator of the effectiveness of KLK1 administration to halt the autoimmune attack or reverse progression of T1D. For example, if KLK1 administration at a certain dose or treatment regimen results in increased C-peptide levels, that dose or treatment regimen may be viewed as being beneficial to the beta cells. Alternatively, if the KLK1 dose or treatment regimen results in a decrease in C-peptide levels, then the T1D may be progressing in the patient resulting in fewer beta cells.

In certain embodiments, for instance, when measuring biomarkers or other indicators of treatment, an “increased” or “decreased” amount or level may include a “statistically significant” amount. A result is typically referred to as statistically significant if it is unlikely to have occurred by chance. The significance level of a test or result relates traditionally to the amount of evidence required to accept that an event is unlikely to have arisen by chance. In certain cases, statistical significance may be defined as the probability of making a decision to reject the null hypothesis when the null hypothesis is actually true (a decision known as a Type I error, or “false positive determination”). This decision is often made using the p-value: if the p-value is less than the significance level, then the null hypothesis is rejected. The smaller the p-value, the more significant the result. Bayes factors may also be utilized to determine statistical significance (see, e.g., Goodman S., Ann Intern Med 130:1005-13, 1999).

In some embodiment, an increase amount or level is about 1.1, 1.2, 1.3, 1.4, 1.5, 2, 2.5, 3, 3.5, 4, 4.5, 5, 6, 7, 8, 9, 10, 15, 20, 25, 30, 40, or 50 or more times (e.g., 100, 500, 1000 times) (including all integers and decimal points and ranges in between and above 1, e.g., 2.5, 2.6, 2.7. 2.8, etc.) the amount produced by no composition (the absence of a KLK1 polypeptide) or a control composition, or the amount produced by the KLK1 composition relative to a control or previous timepoint. A “decreased” or reduced amount or level may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% (including all integers and decimal points and ranges in between) decrease in the amount or level produced by no composition (the absence of an KLK1 polypeptide) or a control composition, or the amount produced by the KLK1 composition relative to a control or previous timepoint.

### Methods for Carrying Out the Invention

### Diabetes and Diagnosis

The disclosure provides methods for treating recent onsent and established type I diabetes, delaying the onset of type 1 diabetes, or patients at increased risk of developing type I diabetes. A method comprises administering a therapeutically effective amount of a KLK1 polypeptide to a patient. In certain embodiments, the KLK1 polypeptide has serine protease activity and/or anti-diabetic activity and has at least about 80% 85%, 90%, 91%, 92%, 93%, 94%, 95%, 96%, 97%, 98%, 98.5%, 99%, or 99.5% amino acid sequence identity with a reference sequence, such as the full length, propeptide or mature KLK1 having a sequence of the corresponding sequence of SEQ ID NO:2.

In some embodiments, a biomarker is measured prior to treatment to identify patients at increased risk of developing type 1 diabetes. In other embodiments, a method further comprises detecting a biomarker to assess effectiveness of a treatment. In some embodiments, a biomarker indicative of an increased risk of developing type 1 diabetes comprises a HLA biomarker, antibodies against insulin, islets, or the enzymes glutamic acid decarboxylase (GAD) and protein tyrosine phosphatase IA2 (also known as ICA512). In certain embodiments, a biomarker useful to assess effectiveness of treatment includes fasting blood glucose, level of ketone bodies, C-peptide levels, IDO levels (e.g., IDO mRNA levels in splenic DCs), and HbA1c levels.

Type 1 diabetes (T1D) is usually first diagnosed in children, teenagers, and young adults when the pancreas no longer makes insulin. In Type 1 diabetes, an autoimmune reaction destroys, functionally impairs, or depletes pancreatic beta-cells. In contrast, Type 2 diabetes is usually diagnosed in later adulthood. Pathologically, type 2 diabetes (T2D) is characterized by an individual that is not able to make enough insulin or is resistant to the insulin produced by the beta cells. Latent Autoimmune Diabetes of Adults (LADA) is an autoimmune T1D that manifests in adults, and LADA patients may not require insulin injections initially as the diabetes develops more gradually. Otherwise LADA has the same etiology as described for T1D in children and teenagers in that an autoimmune reaction results in the destruction of beta cells, which then results in development of insulin dependent diabetes.

Diabetes can be diagnosed by various means. For instance, a fasting plasma glucose (FPG) test measures blood glucose in a person who has not eaten anything for at least 8 hours. The FPG test is the preferred test for diagnosing diabetes because of its convenience and low cost. An oral glucose tolerance test (OGTT) measures blood glucose after a person fasts at least 8 hours and 2 hours after the person drinks a glucose-containing beverage. A random plasma glucose test, also called a casual plasma glucose test, measures blood glucose without regard to when the person being tested last ate. These tests, along with an assessment of symptoms, is used to diagnose diabetes. Test results indicating that a person has diabetes should be confirmed with a second test on a different day.

A random, or casual, blood glucose level of 200 mg/dL or higher, plus symptoms of increased urination, increased thirst, and/or unexplained weight loss, can mean a person has diabetes. Other symptoms can include fatigue, blurred vision, increased hunger, and sores that do not heal. A physician can check the person's blood glucose level on another day using the FPG test or the OGTT to confirm the diagnosis.

The above tests may determine if a patient is suffering from diabetes, but not necessarily whether the patient has type 1 diabetes or type 2 diabetes. Type 1 diabetics tend to be children or young adults and be non-overweight, whereas type 2 diabetic patients tend to be overweight adults. T1D tends to have an acute onset, with symptoms arising within weeks to a few months, whereas T2D tends to have a gradual onset. To differentiate between the two types of diabetes, additional testing is required. Patients with T1D will have low to no detectable insulin and C-peptide, whereas T2D patients tend to have normal to high insulin and C-peptide levels. T1D patients may also present with ketosis or ketoacidosis whereas T2D patients typically do not have ketosis at diagnosis.

Gestational diabetes can also be diagnosed based on plasma glucose values measured during an OGTT, preferably by using 100 grams of glucose in liquid for the test. Blood glucose levels are checked four times during the test. If blood glucose levels are above normal at least twice during the test, the woman is diagnosed as having gestational diabetes. Above-normal results for the OGTT for gestational diabetes are indicated by 95 mg/dL at fasting, >180 mg/dL at 1 hour, >155 mg/dL at 2 hours, and >140 mg/dL at 3 hours (when using 100 g glucose).

An HbA1c test, which measures the level of glycated hemoglobin in the blood, can also be used to test for diabetes. This blood test shows the average amount of glucose in blood during the past 2 to 3 months. For a diagnosis of diabetes, an HbA1c level of 6.5% or higher is required. Subjects who with an HbA1c of 5.7% to 6.4% are considered at an increased risk of developing diabetes.

One patient population that would be treated with KLK1 of the instant invention are “increased risk patients,” also known as pre-diabetic patients or patients that do not have T1D but are at an increased risk of developing T1D. In some embodiments, a method comprises identifying patients at increased risk of developing type 1 diabetes by detecting a biomarker associated with the increased risk. Such increased risk patients who eventually develop type 1 diabetes may have immune biomarkers in their blood such as antibodies against insulin, islets, beta cells, or the enzymes glutamic acid decarboxylase (GAD) and protein tyrosine phosphatase IA2 (also known as ICA512). By measuring these biomarkers and conducting metabolic tests, the risk can be gauged for developing type 1 diabetes. Tests include, for example:
Blood tests for antibodies to components of the insulin-producing islet cells and to insulin itself. Risk increases with the number of positive antibody tests.
A blood test for specific genes that may make individuals more or less susceptible to type 1 diabetes.
A test that measures the amount of insulin produced in response to an intravenous injection of insulin. A decreased first phase insulin response puts a person at increased risk for developing diabetes.
A test that measures fasting blood glucose levels and glucose tolerance. People with prediabetes have impaired fasting glucose or impaired glucose tolerance, or both.

In some embodiments of the instant invention, KLK1 may be administered to increased risk patients, or patients at increased risk for development of type 1 diabetes. In some aspects, the KLK1 may be administered until a biomarker or biomarkers present in increased risk patients improves or decreases such that the patient is no longer considered an increased risk patient. Optionally, in such instances, administration of KLK1 may continue at a lower dosage amount and/or dosage frequency.

There are several investigations aimed at further identifying populations at increased risk for developing Type 1 diabetes, including (ClinicalTrials.gov Identifier: NCT00649246) and (ClinicalTrials.gov Identifier: NCT01042301). The goal is to identify other biomarkers and further quantitate biomarkers for increased risk patients for developing Type 1 diabetes by looking at genetic risks (biomarkers, etc) and other variables. A patient who has not yet developed Type 1 diabetes, is assigned a “risk assessment” score from 1 to 100. The closer to 100, the more likely the patient will develop Type 1 diabetes. The field for identifying increased risk patients is continuing to evolve, but sufficient information has been determined to date that several treatments described in the background herein are targeted to patients with increased risk for development of T1D.

Type 1 diabetes is a polygenic disease, meaning many different genes contribute to its expression. Several alleles of HLA-DQB1 are associated with an increased risk of developing type 1 diabetes. The locus also has the genetic name IDDM1 as it is the highest genetic risk for type 1 diabetes. The DQB1*0201 and DQB1*0302 alleles of IDDM1, particularly the phenotype DQB1*0201/*0302 has an increased risk of developing type 1 diabetes. Other alleles of the IDDM1 gene that increase the risk for T1D include DRB1 0401, DRB1 0402, DRB1 0405 and DQA 0301. The risk is partially shared with the HLA-DR locus (DR3 and DR4 serotypes). There are also variants that appear to be protective. Increased risk patients thus include patients with certain genetic predisposition for T1D.

Environmental factors can influence development of T1D. A study showed that for identical twins, when one twin had T1D, the other twin only had T1D 30%-50% of the time, despite having exactly the same genome. This suggests that environmental factors, in addition to genetic factors, can influence disease prevalence. Other indications of environmental influence include the presence of a 10-fold difference among Caucasians living in different areas of Europe, and a tendency to acquire the incidence of the disease of the destination country for people who migrate. Patients with a certain genetic background and living in certain environments may be deemed patients with increased risk for development of T1D.

One theory proposes that type 1 diabetes is a virally triggered autoimmune response in which the immune system attacks virus infected cells along with the beta cells in the pancreas. The Coxsackie virus family or Rubella virus have been implicated. This vulnerability is not shared by everyone, for not everyone infected by the suspected organisms develops type 1 diabetes. Patients with a certain genetic background and having been exposed to certain viruses may be deemed patients with increased risk for development of T1D.

There is a growing body of evidence that diet may play a role in the development of type 1 diabetes, through influencing gut flora, intestinal permeability, and immune function in the gut; wheat in particular has been shown to have a connection to the development of type 1 diabetes.

An animal model of patients at increased risk of developing type I diabetes is the Non-Obese Diabetic (NOD) mouse. Female mice spontaneously develop type 1 diabetes at a rate of 60 to 80% by age 20 weeks due to an autoimmune reaction against their beta cells. Male mice also develop T1D though at a lower rate (20 to 30%). Similar to human increased risk patients, NOD mice also display immune dysfunction and other symptoms of autoimmune disease prior to development of T1D, as described in: The NOD Mouse: A Model of Immune Dysregulation (Mark S. Anderson and Jeffrey A. Bluestone, Annu. Rev. Immunol. 23:447-85, 2005). Additionally, again similar to human increased risk patients, NOD mice also have dysfunction in glucose control prior to development of T1D, as described in: Glucose Homeostasis in the Nonobese Diabetic Mouse at the Prediabetic Stage (Abdelaziz Amrani, et al. Endocrinology. 139: 1115-1124, 1998). As such, NOD mice are a good animal model for increased risk patients.

Patients that are diagnosed with T1D first present with classic symptoms of polyuria, polydipsia, polyphagia, and weight loss. Typically, at diagnosis, between 80-90% of their insulin producing beta cells have already been destroyed. The first few weeks after diagnosed with T1D is considered the “Honeymoon Phase” or “recent onset” as patients still have about (e.g., at least about, less than about) 5-20% or 10-20% of their pancreatic beta cells and still maintain an ability to produce insulin. Because the patients still have remaining beta cells, they may have low but detectable fasting C-peptide level, wherein C-peptide levels increase during mixed meal tolerance test with a minimal stimulated value of ≧0.2 pmol/mL (indicative of remaining beta cells producing insulin). The patients may also have increased antibodies against islets, increased antibodies against glutamic acid decarboxylase (GAD), increased antibodies against protein tyrosine phosphatase (IA2/ICA512), increased circulating T cells that react with beta cell antigens, increased insulitis, increased inflammation of the pancreas. Such patients would have decreased regulatory T cells (Tregs) (CD4+ cells that are also CD25+/Foxp3+), increased HbA1c levels, decreased C-peptide levels, and decreased IDO (Indoleamine-pyrrole 2,3-dioxygenase) levels, relative to a healthy control or other reference standard.

Many proposed therapies described in the background herein are aimed at treating T1D patients in the honeymoon phase or recent onset, with the goal of halting or ameliorating the autoimmune attack on beta cells and allowing some insulin production. T1D patients that are in the honeymoon phase or recent onsent of T1D may also be treated with KLK1 of the invention to attenuate the autoimmune reaction. The effectiveness of KLK1 treatment may be monitored by measuring biomarkers of autoimmune reaction against the beta cells, including, for example, decreases in autoantibodies, increases in C-peptide levels, increases in IDO mRNA or expression, increases in regulatory T cells, among other biomarkers, and therapy may be optionally adapted accordingly (e.g., upon improvement of one or more biomarkers), for instance, by maintaining or increasing the dosage until improvement of the biomarker relative to an earlier time point, or by reducing the dosage of KLK1 or terminating therapy upon improvement of the biomarker relative to a healthy control or other reference marker.

Following the Honeymoon Phase, the type 1 diabetes will typically progress to established diabetes where patients require insulin injections to control their blood glucose levels and prevent ketoacidosis. Such patients but may still have some detectable C-peptide levels indicating the presence of residual beta cells. However, unlike patients with functional beta cells wherein C-peptide levels increase following a meal, the C-peptide levels will have minimal to no increase (≧0.2 pmol/mL) following mix meal stimulation. T1D patients that are in the established stage of T1D may also be treated with KLK1 of the invention to attenuate the autoimmune reaction. The effectiveness of KLK1 administration to patients in the post Honeymoon Phase or with established diabetes the may be monitored by measuring biomarkers of autoimmune reaction against the beta cells including decreases in autoantibodies, increases in C-peptide levels, increases in IDO mRNA or expression, increases in regulatory T cells, among other biomarkers described herein.

In such established T1D patients, recent evidence suggests residual beta cells still exist but in a quiescent state such that the beta cells to not express insulin or many beta cell specific surface antigens. Attenuation of the autoimmune reaction in such patients has resulted in these residual beta cells emerging from their quiescent state, as evidenced by detectable C-peptide levels in patients decades after development of T1D. In one embodiment, administration of KLK1 polypeptide to established T1D patients may result in attenuation of the autoimmune reaction against the beta cells. Any residual beta cells in the established T1D patients may then emerge from their quiescent state and begin production of insulin, as evidenced by detection of circulating C-peptide and/or decrease in the amount of insulin required to be injected to maintain blood glucose levels. Changes in other biomarkers described herein may also be monitored to determine the effectiveness of KLK1 treatment.

In LADA diabetic patients, the onset of diabetes is slower. In some embodiments, administration of KLK1 polypeptide to patients with LADA results in attenuation of the autoimmune reaction against beta cells, resulting in increased C-peptide levels and/or decrease in the amount of insulin required to be injected to maintain blood glucose levels. Changes in other biomarkers described herein may also be monitored to determine the effectiveness of KLK1 treatment.

In some embodiments, the dosage amount and/or frequency of a KLK1 polypeptide can be reduced or therapy terminated if improvement in the biomarker relative to a control is observed. In some aspects, the dosage amount and/or frequency of a KLK1 polypeptide can be reduced or therapy terminated if improvement in the biomarker relative to a control is maintained over a defined period of time, for instance, about 1, 2, 3, 4, 5, 6, 7, 8, 9, 10, 11, 12, 13, 14, 16, 17, 18, 19, 20, 21 weeks, or months. For example, in certain embodiments the dosage can be decreased by about 1.1×, 1.2×, 1.3×, 1.4×, 1.5×, 1.6×, 1.7×, 1.8×, 1.9×, 2×, 2.5×, 3×, 3.5×, 4×, 4.5×, 5×, 6×, 7×, 8×, 9×, 10×, 15×, 20× or more, relative to the previous dosage. Merely by way of illustration, the dosage frequency can be decreased by about 1, 2, 3, 4, 5 or fewer dosages per day, and/or 1, 2, 3, 4, 5, 6, 7, 8, 9, 10 or fewer dosages per week, relative to the previous dosing schedule. As noted above, the dosage amount can be decreased separately or in combination with the dosage frequency, and vice versa, optionally until a desired level or range of one or more biomarkers (e.g., C-peptide levels, IDO levels) or other treatment indicators is achieved.

Even after reduction or termination of KLK1 therapy due to improvement (e.g., increase or decrease) in one or more biomarkers, such biomarkers can continue to be monitored in the patient. In these and related embodiments, if the earlier improvement in biomarkers is observed to reverse course, then KLK1 dosage (amount/frequency) can be increased as described herein, and/or KLK1 treatment may be resumed.

Methods for administration of KLK1 of the invention to a patient in need thereof include oral and parenteral administration. Examples of parenteral administration include subcutaneous, intramuscular, intravenous, intra-arterial, and intraperitoneal administration. Compositions of the present invention also may include therapeutically effective amounts of product in combination with acceptable diluents, excipients or carriers. In certain embodiments, it is preferred that the compositions are administered parenterally. The specific route of administration may depend, for example, on the medical history of the patient, including any perceived or anticipated side effects using KLK1. While not meant to limit the scope of the invention, it is believed that parenteral administration allows for administration of a lower dose of the medication.

The administration may be by continuous infusion (using, e.g., minipumps such as osmotic pumps), or by injection using, e.g., intravenous or subcutaneous means. In one embodiment, KLK1 is administered subcutaneously. The administration can also be as a single bolus or by slow-release depot formulation.

KLK1 to be used in a therapy is formulated and dosed in a fashion consistent with good medical practice, taking into account the specific condition being treated, the clinical condition of the individual patient (especially the side effects of treatment with KLK1), the site of delivery of KLK1, the method of administration, the scheduling of administration, and other factors known to practitioners. The “effective amount” of KLK1 for purposes herein (including an amount to counteract, e.g., autoimmune destruction of beta cells) is thus determined by such considerations.

The term “therapeutically effective amount” refers to a nontoxic but sufficient amount of KLK1 polypeptide effective to “alleviate” or “treat” recent onset type 1 diabetes or established type 1 diabetes or LADA in a subject, which can be a mammal (e.g., humans, cats, dogs). Generally, alleviation or treatment of a disease or disorder involves the lessening of one or more symptoms, biomarkers, or medical problems associated with the disease or disorder, such as the autoimmune reaction against beta cells.

The decrease in autoimmune reaction may be measured, for example, by modulation of biomarkers. For example, a decrease in the number of antibodies against insulin, islets, or the enzymes glutamic acid decarboxylase (GAD) and protein tyrosine phosphatase IA2 (also known as ICA512) can be used to assess the effectiveness of treatment comprising KLK1 administration. Other measurements of the autoimmune reaction are a decrease in the levels of circulating T cells that react to beta cell antigens, a decrease in insulitis or inflammation of the pancreas, or an increase in suppressor (regulatory) T cells (Tregs) (CD4+ cells that are also CD25+/Foxp3+). Several studies have demonstrated that human patients with T1D have significantly decreased frequencies of functional regulatory T cells (Treg), defined by their co-expression of CD4 and CD25 as well as the transcription factor FOXP3. Another measurement is a decrease in the number of patients (or animal models) needing to have insulin to moderate their blood glucose levels. Other biomarkers may also be measured. In the above examples, references to increase or decrease can be relative to the level in the patient prior to treatment, relative to an earlier stage of treatment, or relative to a health control or other references standard used in the art.

Other biomarkers for assessing the effectiveness of a KLK1 treatment include determining whether fasting blood glucose, HbA1c, or ketone bodies are reduced relative to the level in the patient prior to treatment, or relative to an earlier stage of treatment.

Certain biomarkers for assessing the effectiveness of a KLK1 treatment include determining whether C-peptide levels and/or IDO levels are increased relative to the level in the patient prior to treatment, or relative to an earlier stage of treatment. The levels of these and related biomarkers can be measured by protein, mRNA, or both, according to routine techniques in the art, such as ELISA, PCR, qPCR, etc. In specific embodiments, C-peptide levels can be determined, for example, by performing an ELISA on blood, serum or other tissue sample. In particular embodiments, IDO levels can be determined, for example, by measuring mRNA levels, optionally in specific cell populations such as splenic DCs.

The terms “treating” or “treatment” is an approach for obtaining beneficial or desired clinical results. Herein, beneficial or desired clinical results include, but are not limited to, alleviation or inhibition of symptoms, biomarkers predictive for development of type 1 diabetes, diminishment of extent of disease, stabilization (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” is an intervention performed with the intention of preventing the development or altering the pathology of a disorder. Measurements of treatment in patients in the honeymoon phase of T1D, or recent onset T1D or LADA, include increase production of endogenous insulin in the patient, as measured by insulin levels or C-peptide levels in the blood, improved or normal fasting blood glucose levels (euglycemia) or improved response to a glucose tolerance test. Other measurements include, but are not limited to, decreased autoimmune reaction, as measured by decreases in antibodies against insulin, islets, or the enzymes glutamic acid decarboxylase (GAD) and IA2, or increases in Treg cells. Measurements of treatment in increased risk patients include, but are not limited to, continued production of endogenous insulin in the patient, as measured by insulin levels or C-peptide levels in the blood, euglycemia and normal response to a glucose tolerance test, low or no detectable antibodies against insulin, islets, or the enzymes glutamic acid decarboxylase (GAD) and IA2. Another measurement is a decrease in the number of patients (or animal models) needing to have insulin to moderate their blood glucose levels. In the above examples, references to increase or decrease are relative to the level in the patient prior to treatment, or relative to an earlier stage of treatment.

The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

Generally, an effective amount of KLK1 administered parenterally per dose includes 0.01 U/kg/day to about 1000 U/kg/day of patient body weight for a 60 kg human, although, as noted above, this is subject to therapeutic discretion. As an example, a dose may be about 0.1 U/kg/day to 100 U/kg/day, or 0.2 U/kg/day to 30 U/kg/day of patient body weight. A key factor in selecting an appropriate dose is an obtained result, as measured for example in treating the onset of diabetes of extending the diabetic Honeymoon Phase, normalized blood glucose levels, C-peptide levels or levels of another relevant biomarker, decreased use of insulin or other glucose moderating drugs.

Tissue kallikrein-1 polypeptides and compositions of the present invention may be lyophilized or made into tablets. Standard diluents such as human serum albumin are contemplated for pharmaceutical compositions of the invention, as are standard carriers such as saline.

Tissue kallikrein-1 products of the present invention may be useful, alone or in combination with other factors or drugs having utility in immunomodulation, specifically drugs or substances described in the background that may halt autoimmune reactions.

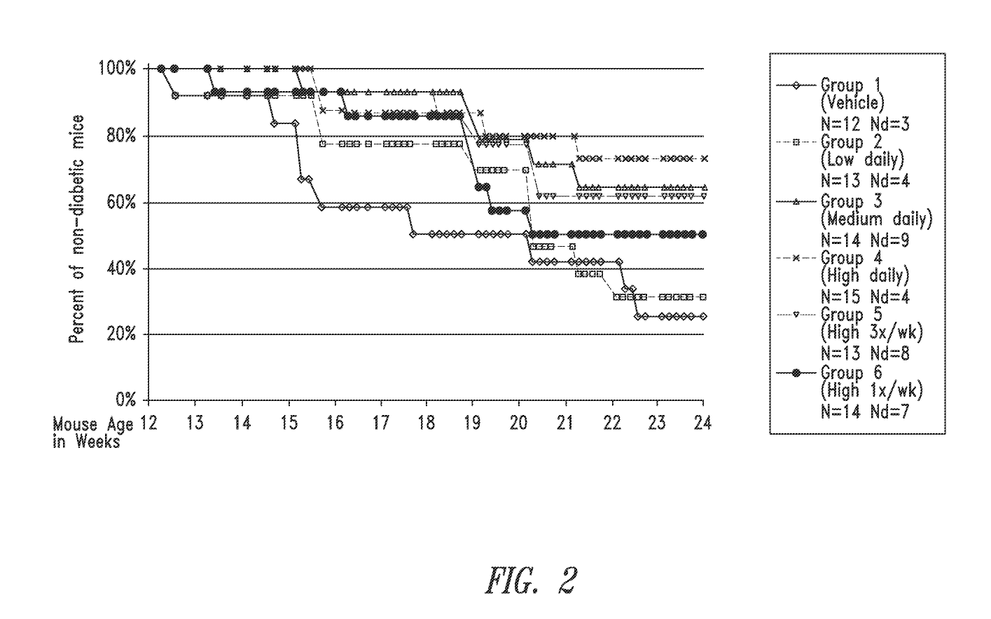

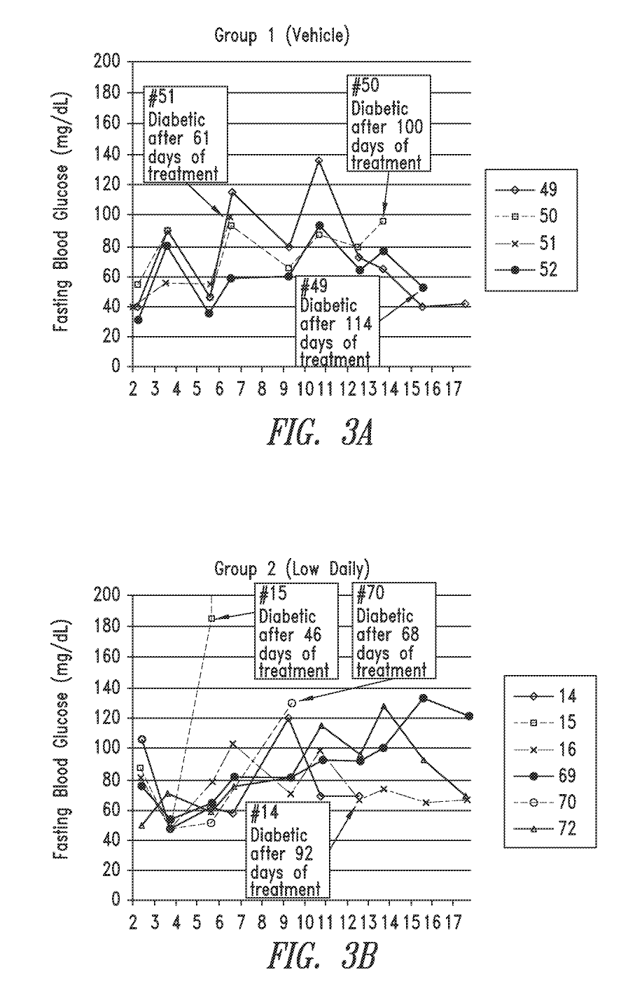

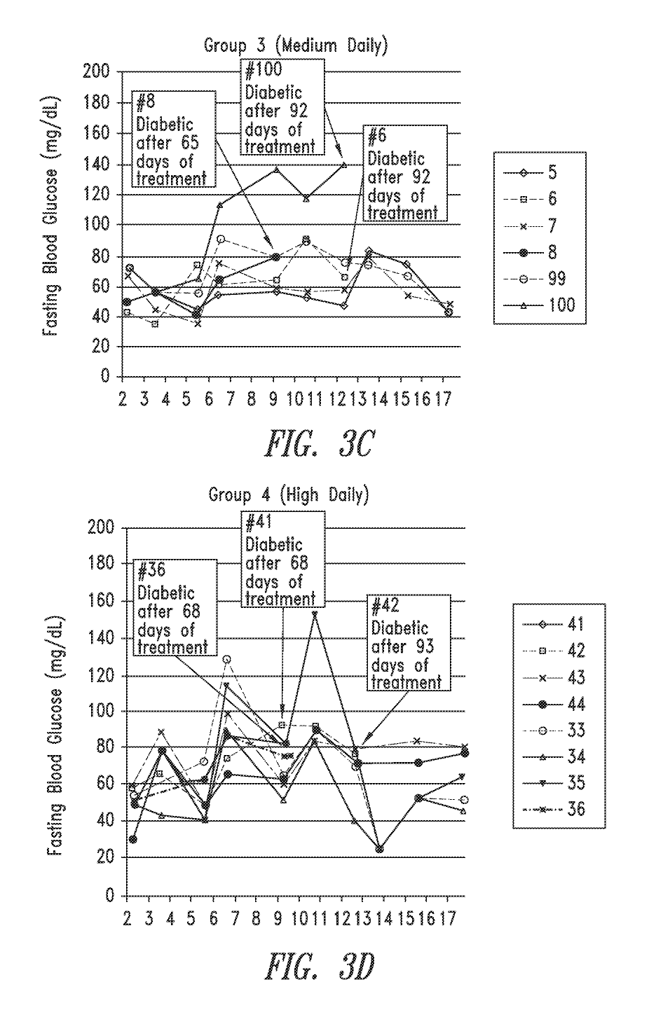

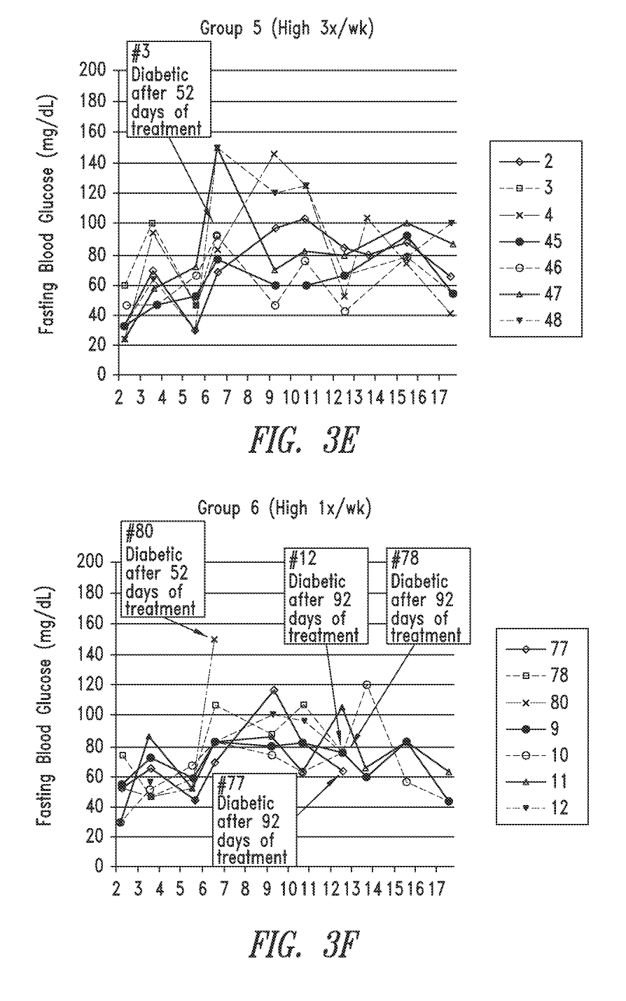

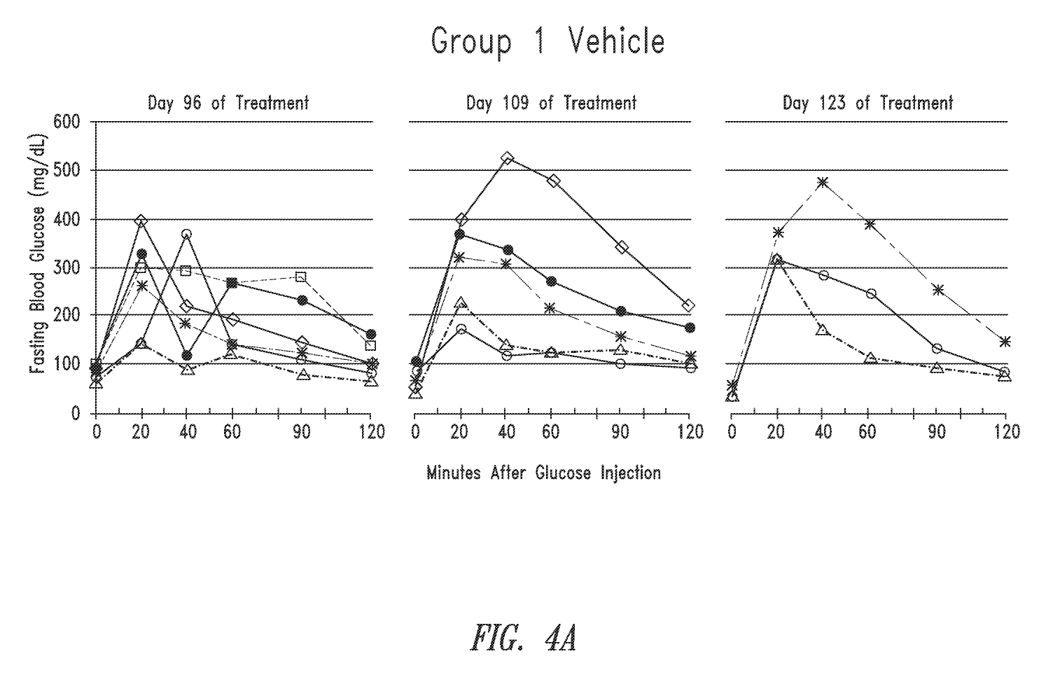

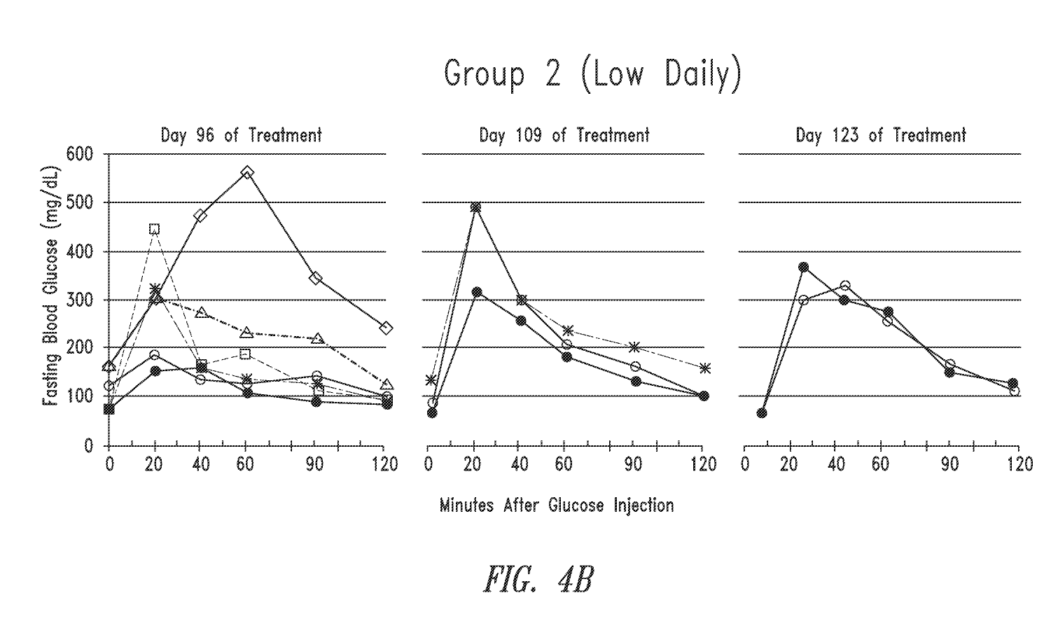

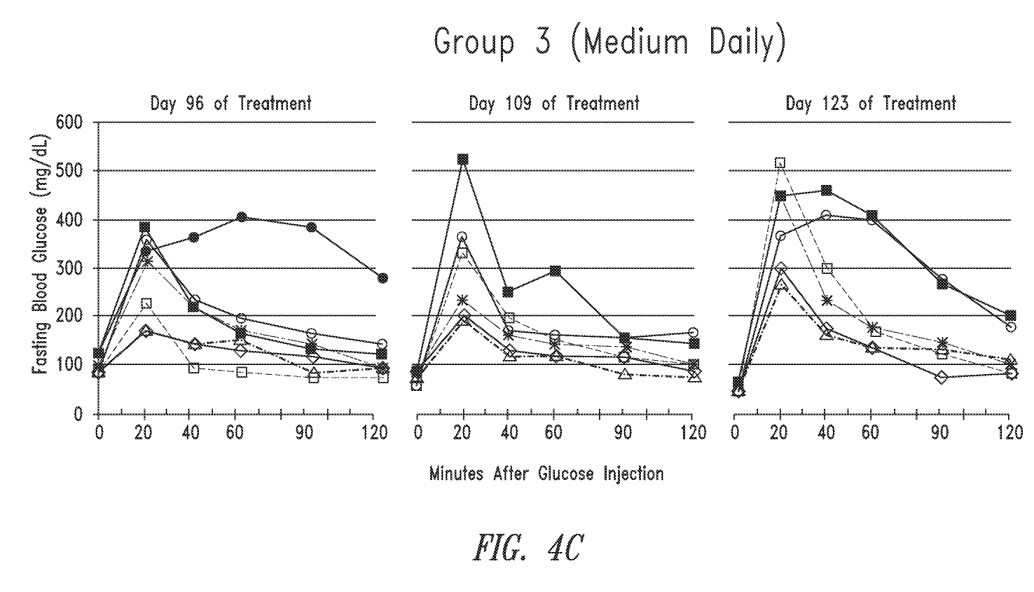

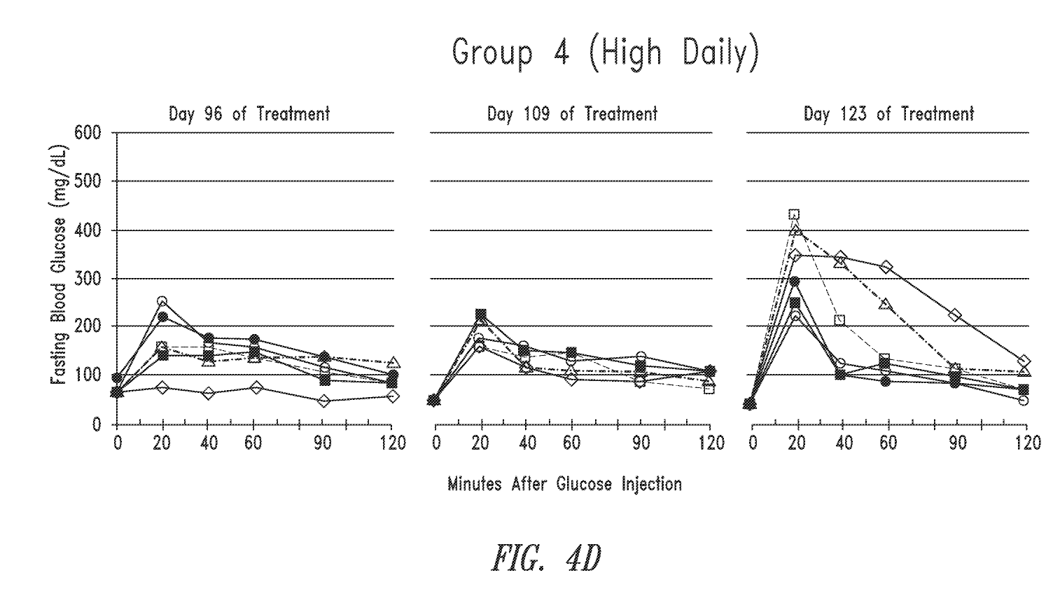

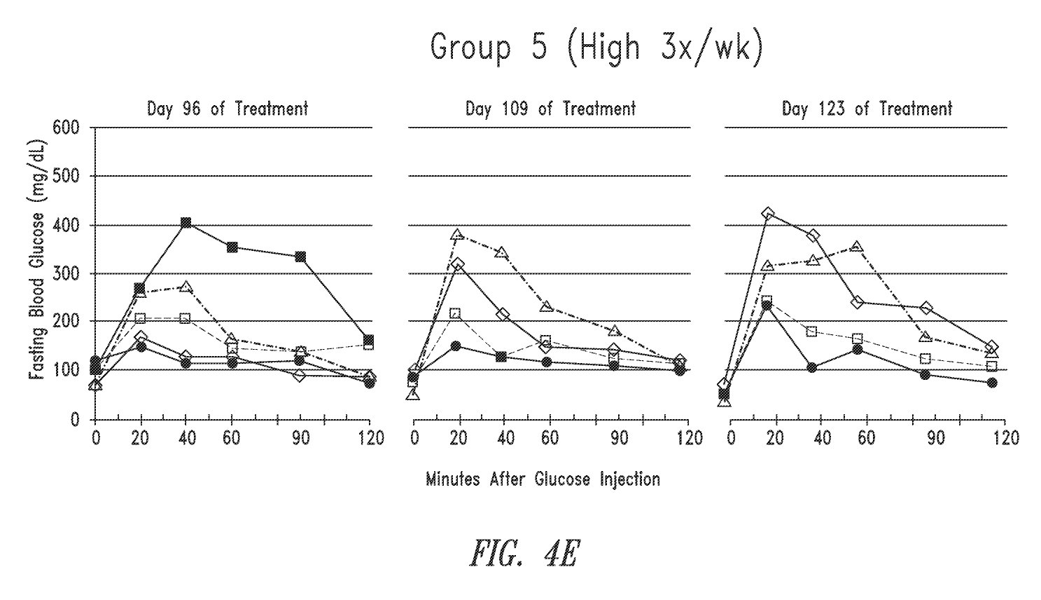

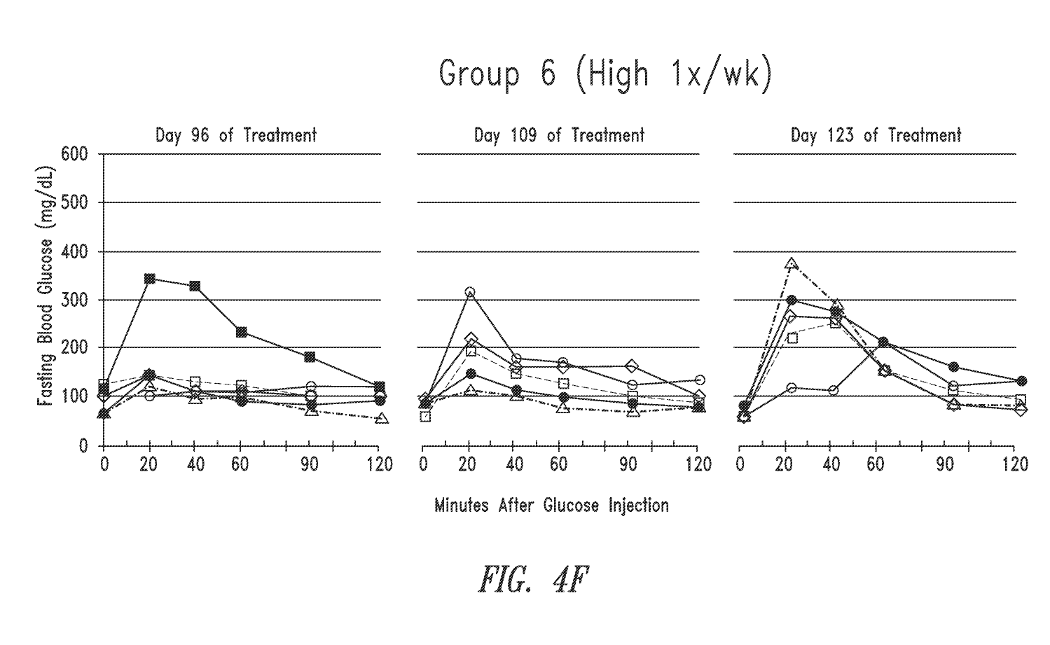

In embodiments, a therapeutically effective dose may be selected based on the ability of the dose to maintain fasting blood glucose and glucose tolerance within normal range, and/or to increase the number of CD4 positive, CD25 positive, and Foxp3 positive cells in the spleen. In some embodiments, different dosing schemes have been studied in the NOD mouse system. In a specific embodiment, a dosing scheme of once per day of a high dose of KLK1 was effective to delay onset of Type 1 diabetes, and modulate fasting blood glucose and glucose tolerance. (See FIGS. 3 and 4). A dosing scheme of a high dose once per day as well as a high dose 3 times per week were effective in increasing the percentage of protective CD4-positive, CD25-positive, Foxp3 lymphocytes in the spleen. (see FIG. 6). In particular embodiments, the dosage is applied once per day, and/or three times per week. Certain embodiments include methods of optimizing a dosing scheme by administering a KLK1 polypeptide, and measuring (before, during, and/or after a given dose) the level of one or more biomarkers (e.g., C-peptide, IDO) or other indicators of treatment (e.g., blood glucose levels, reduced usage of insulin or other diabetic drug), and adjusting the dosing scheme according to the measurement(s), usually when the measurement(s) fall outside of a desired level or range. For instance, if C-peptide and/or IDO levels are below a desired level or range, the frequency of the dosages and/or the dosage amount may be increased, optionally until the desired range or level is achieved.

The dosage amount can be increased, merely by way of example, by about 1.1×, 1.2×, 1.3×, 1.4×, 1.5×, 1.6×, 1.7×, 1.8×, 1.9×, 2×, 2.5×, 3×, 3.5×, 4×, 4.5×, 5×, 6×, 7×, 8×, 9×, 10×, 15×, 20× or more, relative to the previous dosage. The dosage frequency can be increased, merely by way of illustration, by about 1, 2, 3, 4, 5 or more dosages per day, and/or 1, 2, 3, 4, 5, 6, 7, 8, 9, 10 or more dosages per week, relative to the previous dosing schedule. As noted above, the dosage amount can be increased separately or in combination with the dosage frequency, and vice versa, optionally until a desired level or range of one or more biomarkers (e.g., C-peptide levels, IDO levels) or other treatment indicators is achieved.

According to the FDA Guidance for Industry; Estimating the Maximum Safe Starting Dose in Initial Clinical Trial for Therapeutics in Adult Healthy Volunteers (July 2005), Appendix D: Converting animal doses to human equivalent doses. A human equivalent dose is 1/12 the mouse dose. A Mouse dose 0.08 U of KLK1 per mouse is described here. The mice used in the instant study weighted approximately 30 grams, equating to a mouse dose of 2.7 U/kg. The human equivalent dose ( 1/12) would be approximately 0.22 U/kg. A mouse dose 0.4 U of KLK1 per mouse equates to approximately 13.3 U/kg in mice. The human equivalent dose would be approximately 1.11 U/kg. A mouse dose 2 U of KLK1 per mouse equates to approximately 66. 7 U/kg in mice. The human equivalent dose would be approximately 5.55 U/kg. Such dosing extrapolation from mouse to human is provided as an example only, and methods for determining a dose for treatment of humans are described herein throughout.

The dosing described above is based on the enzymatic activity of the KLK1, which may be determined by the methods described herein. The enzymatic activity or specific activity of any batch of KLK1 that is manufactured may vary.

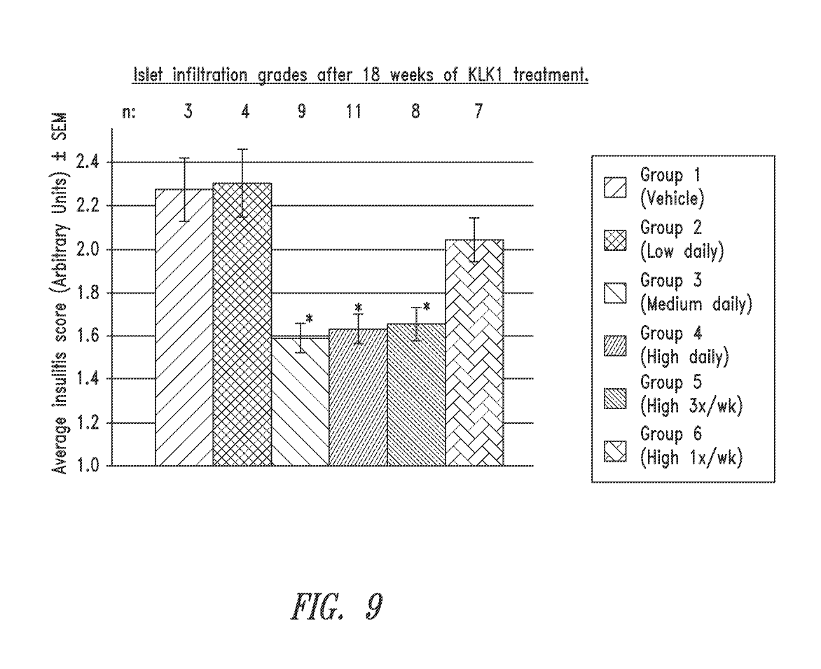

To determine if a dose of KLK1 is effective in treating a patient, either in terms of the amount (number of units administered to a patient) of KLK1 administered or the frequency of administration, several biomarkers may be measured. The following biomarkers are described as examples, and are not intended to be an exhaustive list. An effective dose of KLK1 may increase the numbers of T-regulatory cells in the spleen, specifically CD4+/CD25+/Foxp3+ cells in the spleen, as observed in FIG. 6 and related written description. Another endpoint to determine the effective dose is an improvement in a glucose tolerance test, as observed in FIG. 4 and related written description. Another endpoint to determine the effective dose is a decrease in insulitis, as observed in FIG. 9 and related written description, which measured by pancreatic biopsies, or other non-invasive procedures in humans. Another endpoint to determine the effective dose is an increase in IDO expression, as determined by mRNA or protein expression as observed in FIG. 16. In the above examples, references to increase or decrease are relative to the level in the patient prior to treatment, or relative to an earlier stage of treatment.

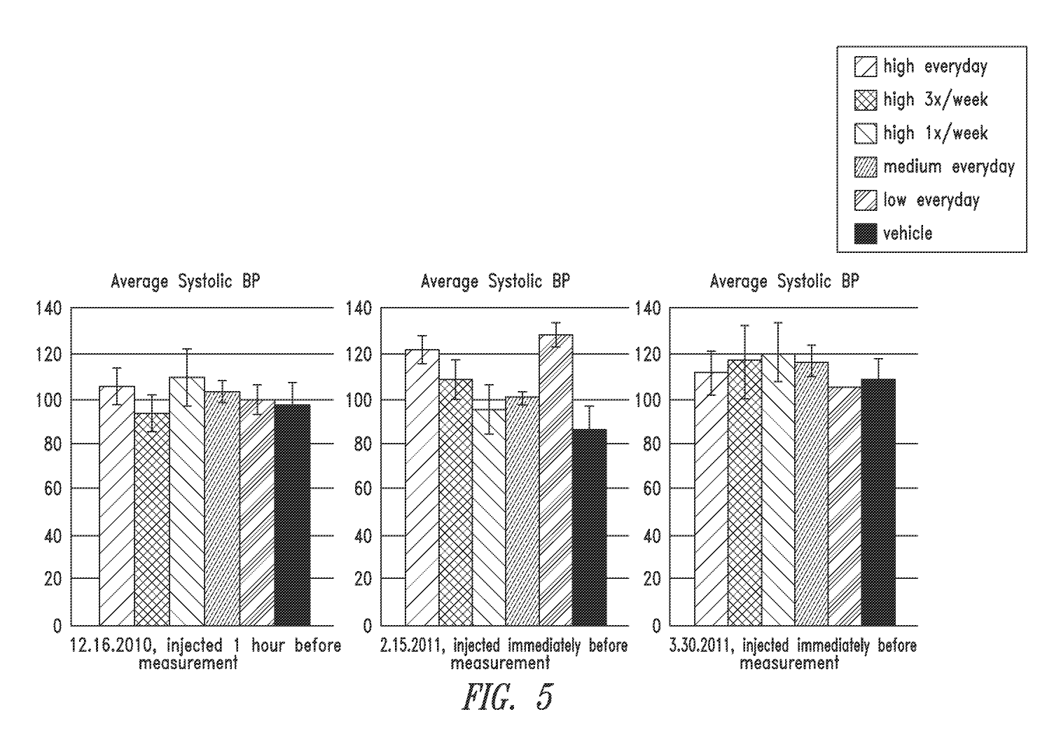

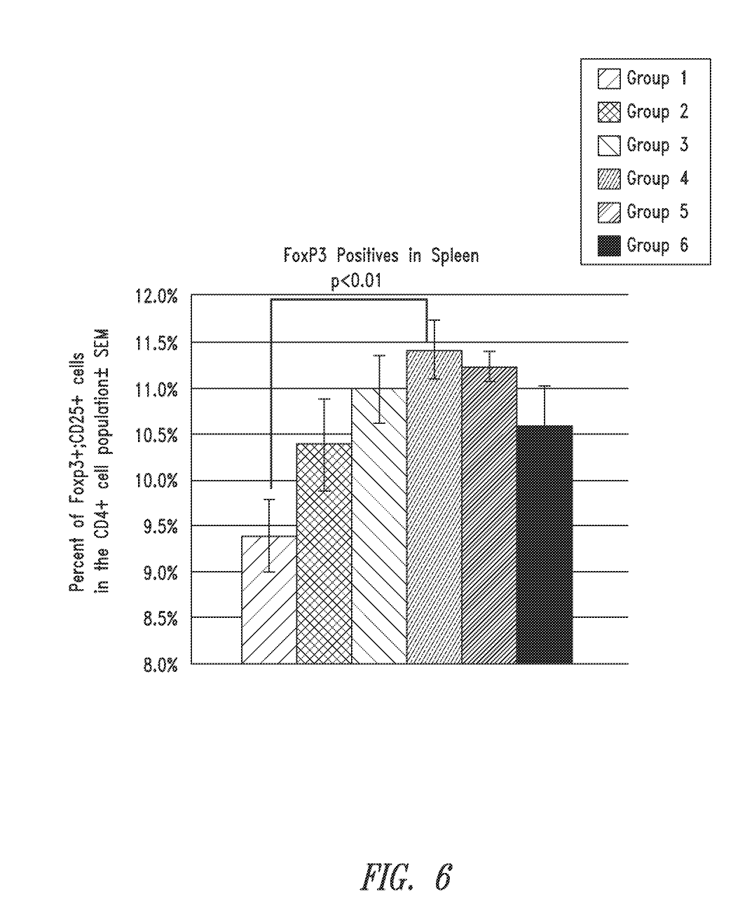

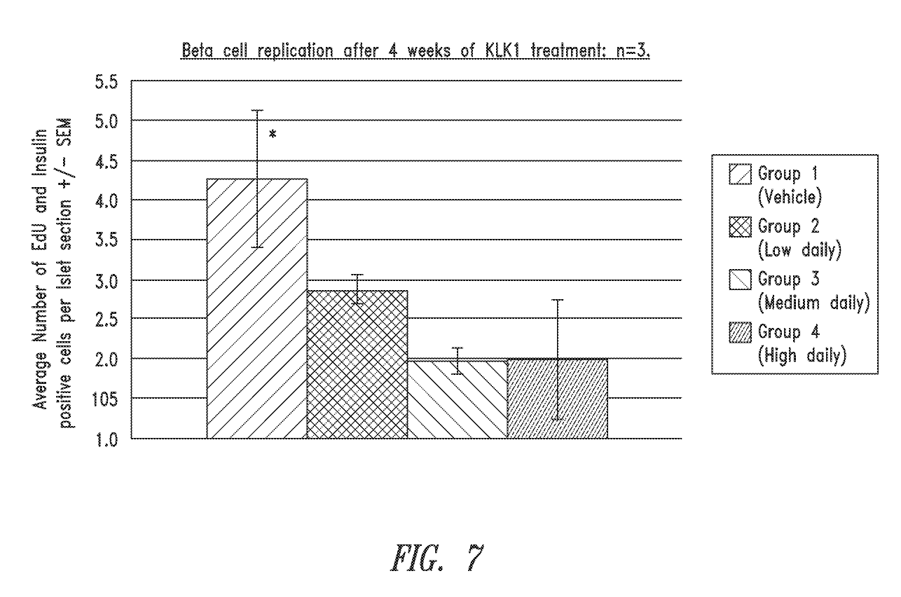

In other embodiments, a therapeutically effective dose is the amount of KLK1 that treats or delay the onset of type I diabetes without adverse side effects on blood pressure and heart rate. As shown in FIG. 5, KLK1 did not impact heart rate or blood pressure when measured immediately after or within 1 hour of injection of KLK1.

Several reports have published that KLK1 levels in diabetic patients are higher than normal. Specifically KLK1 levels and activities are significantly higher in patients with type 1 diabetes than in controls, and KLK1 levels are significantly higher in pregnant women with gestational diabetes than in healthy pregnant women (Miranda et al, Int J Diabetes & Metab. 18: 124-131, 2010). Abnormally high levels of KLK1 in patients with type 1 diabetes and gestational diabetes would suggest that a treatment to further increase KLK1 levels would be counterintuitive. In addition, it was uncertain that KLK1 could be administered to treat a conditions such as type I diabetes without adversely impacting heart rate or blood pressure by causing for example, a drop in blood pressure.

The following examples are presented by way of illustration of the invention and not by way of limitation. Those of skill in the art will readily recognize a variety of noncritical parameters that could be changed or modified to yield essentially similar results.

### EXAMPLES

### Example 1

A cDNA coding for pre-pro-human KLK1, the 262 amino acid residue sequence depicted in SEQ ID NO:2, was purchased from OriGene™ (Rockville, Md., USA). The KLK1 cDNA (Catalogue No. SC122623) is a human cDNA open reading frame clone, cloned into the multi-cloning site of OriGene's pCMV6-XL5 vector, between a cytomegalovirus (CMV) promoter to control transcription of cDNA coding for pre-pro-human KLK1 and a polyadenylation signal. This KLK1 clone was sequenced and, using translation software, translated to reveal Seq ID NO:2. This sequence differed at 2 amino acid residues from the human KLK1 sequence in GenBank (Ref No. NP_{—}002248.1). Specifically, SNPs resulted in an apparent E to Q at amino acid residue 145 of 262, and an apparent A to V position 188 of 262, as depicted in SEQ ID NO:2 all subsequent experiments were performed with KLK1 having the amino acid sequence in SEQ ID NO:2.

The human KLK1 cDNA in the pCMV6-XL5 was transfected into a CHO cell line using the FreeStyle™ MAX CHO Expression System (Invitrogen, Carlsbad, Calif. Catalog no. K9000-20). The kit allowed for transient transfection of vectors into Chinese Hamster Ovary (CHO) cells, growth of the transfected CHO cells in 10 liter culture, and protein expression in defined, serum-free medium. The CHO cells are grown in suspension and transient transfection of the KLK1 vector was performed with the liposome reagent supplied in the kit as per instructions.

Expression and purification of recombinant human KLK1 were performed essentially as described by Hsieng S. Lu et al. (Purification and Characterization of Human Tissue Prokallikrein and Kallikrein Isoforms Expressed in Chinese Hamster Ovary Cells, Protein Expression and Purification, 8:227-237, 1996). Briefly, following transfection and allowing sufficient time for expression of recombinant human KLK1, culture supernatant from the 10 liter culture of CHO cells was harvested by centrifugation followed by 0.2 micron filtration. Clarified supernatant was then concentrated, reacted with trypsin to activate the recombinant human KLK1. Because the transient transfection was performed with the cDNA coding for pre-pro-human KLK1, the recombinant human KLK1 secreted from the CHO cells was in an inactive proprotein form. Therefore, activity assay of cell culture supernatant KLK1 involves an activation step with trypsin digestion. Activation is done with trypsin at 10 nM final concentration for 2 hours at room temperature. After the recombinant human KLK1 was activated, the trypsin was neutralized by addition of Soysbean Trypsin Inhibitor (SBTI) (Sigma).

Following activation of the recombinant human KLK1, ammonium sulphate was added to the supernatant, and it was loaded onto an OCTYL SEPHAROSE® column. The Octyl column elution pool of active KLK1 was further purified by Benzamidine affinity column. Pooled active fractions off the Benzamidine column were then buffer exchanged into DEAE equilibration buffer and polished by DEAE column. Active KLK1 fractions from DEAE were pooled and buffer exchanged into 1×PBS buffer. The final KLK1 bulk drug substance was aliquoted and stored at −20° C.

### Example 2

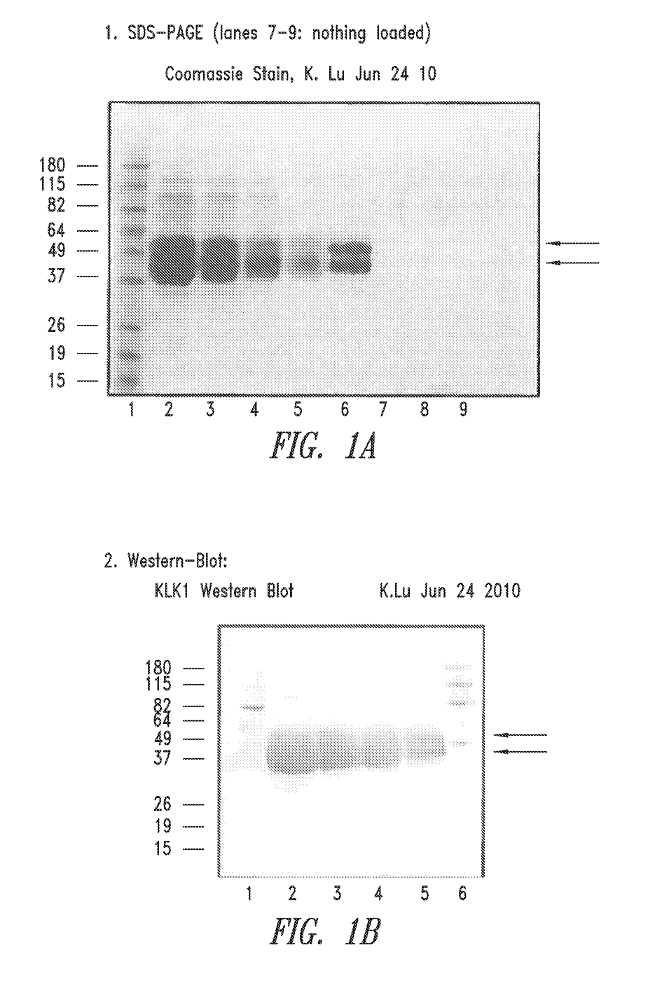

The purified recombinant human KLK1 contained approximately 30% carbohydrate content based on the molecular weight estimated by sodium dodecyl sulphate (SDS) polyacrylamide gel electrophoresis (see FIG. 1A). KLK1 from CHO cells appears as a band having an apparent molecular weight of −40 to 49 kDa; such a broad band may result from different glycosylation forms of KLK1 secreted by CHO cells. For KLK1 expressed in 293 cells, two bands appeared on the SDS-PAGE gel at approximately 40 kDa and 45 kDa. The identity of the bands as human KLK1 was confirmed by Western blot analysis using mouse polyclonal antibody raised against a full-length human KLK1 protein (Catalog #H00003816-B01P, KLK1 purified MaxPab mouse polyclonal antibody (B01P), Abnova Corporation, Walnut, Calif., USA) (see FIG. 1B). The Western blot confirms the results of the SDS-PAGE gel, in that recombinant human KLK1 from CHO cells appears as a band having an apparent molecular weight of 40 to 49 kDa, and KLK1 expressed in 293 cells resolves as two bands at approximately 40 kDa and 45 kDa.

The purity of KLK1 from CHO was visually estimated from the SDS_PAGE gel to be >90% with a final concentration of 1.19 mg KLK1 protein/ml. From the SDS-PAGE gel, it appears CHO produced KLK1 also contains higher molecular weight impurities (˜70-95 kDa) that are not visible in the 293 preparation.

An enzyme activity assay was used to test for activity of recombinant human KLK1 in cell culture supernatants, chromatography fractions during purification and in the final purified product. One fluorogenic substrate suitable for tissue kallikrein-1 measurement of activity is D-val-leu-arg-7 amido-4-trifluoromethylcoumarin (D-VLR-AFC, FW 597.6) (Sigma, Cat #V2888 or Ana Spec Inc Cat #24137.) When D-VLR-AFC is hydrolyzed, the free AFC produced in the reaction can be quantified by fluorometric detection (excitation 400 nm, emission 505 nm according to the catalogue, but alternate excitation and emissions are possible, including excitation 360 nm, emission 460 nm) or by spectrophotometric detection at 380 nm (extinction coefficient=12,600 at pH 7.2).

The measurement of recombinant human KLK1 activity (Units/ml) produced in the CHO cells was determined by comparing the relative activity of recombinant KLK1 to the Kininogenase, Porcine standard acquired from the National Institute for Biological Standards and Control (NIBSC Product No. 78/543). For this standard, the assigned potency is 22.5 international units (IU) per 20 μg ampoule of porcine pancreatic kininogenase. All dosing of NOD mice was based on units of KLK1.

### Example 3

The objective of this experiment was to investigate the anti-diabetic properties of recombinant human KLK1 protein in a non-obese diabetic (NOD) mouse model of Type 1 Diabetes (T1D). Specifically, this experiment examined whether KLK1 treatment of pre-diabetic NOD female mice affects development of spontaneous diabetes, by assessing the dynamics of T1D development, incidence rates and time of disease onset.

Female mice NOD/ShiLtJ were purchased from Jackson Laboratory at 4 weeks of age. All animal procedures were performed in accordance with IACUC policies and under approved protocols. The mice were delivered and allowed to acclimate for 2 weeks, with food and water provided ad libitum. After the 2 week acclimatization period, (at which point the mice were 6 weeks of age) the animals were randomly divided into 6 treatment groups of 12 mice per group. The groups received treatment as described in Table 1 below.

TABLE 1

| | | | | Treatment | Delivery |
|---|---|---|---|---|---|
| Group # | KLK1-Dose | | Description | Frequency | Route |
| 1 | 0 | | Negative Control | 1 x a day | i.p. |
| | | | (Vehicle) | | |
| 2 | 0.08 | Units | low daily | 1 x a day | i.p. |
| 3 | 0.4 | Units | medium daily | 1 x a day | i.p. |
| 4 | 2 | Units | high daily | 1 x a day | i.p. |
| 5 | 2 | Units | high 3x/wk | 1 x every 3 days | i.p. |
| 6 | 2 | Units | high 1x/wk | 1 x every 7 days | i.p. |

Based on previous studies to established time-course, 60 to 80% of NOD female mice were expected to spontaneously develop T1D, with most of the T1D developing between age 18-20 weeks. The mice received KLK1 treatment for 18 weeks, or until the development of overt diabetes (newly-diabetic animals were immediately sacrificed). Since mice were 6 weeks of age at the beginning of treatment, non-diabetic mice were 24 weeks of age at the end of the treatment, past the age when they would be expected to develop T1D.

During the treatment period, all mice were subjected to urinalysis every other day to monitor for urine glucose levels using Diastix™ Reagent Strips (Bayer™), which develops a colorimetric reaction upon contact with one drop (approximately 50 μl) of urine. If urinalysis revealed high levels of glucose in urine (>250 mg/dL) in any particular animal, on any occasion, the animal was subjected to daily blood glucose testing. If non-fasting blood glucose levels were higher than 250 mg/dL for two consecutive days, the mouse was considered diabetic and was immediately sacrificed. In additional, each animal's 12 hour fasting blood glucose levels (no food for 12 hours prior to testing, water provided ad libitum) were assessed twice a week. Blood glucose levels throughout the entire experimental protocol were determined by analysis of 25 μl of blood, obtained after tail vain puncture, using an Ascensia ELITE™ one-touch blood glucose meter (Bayer).

### Results

Treatment with Recombinant Human KLK1 Protects Against Development of Type 1 Diabetes in NOD Mice.

The dynamics of T1D onset in the mice, as described above, in the various treatment groups was carefully recorded and analyzed, and the results are depicted using Kaplan-Meyer curves (see FIG. 2). NOD mice in Group 1 treated with vehicle only (negative control) spontaneously developed type 1 diabetes at the expected rate of approximately 75% by 24 weeks of age. Remarkably, mice in Group 4 dosed at the highest dose of 2.0 U of KLK1 daily, only 27% of mice developed diabetes at 24 weeks of age. In Groups 3 and 5 (medium daily and high 3×/wk), receiving 0.4 U of KLK1 daily and 2 U of KLK1 every 3 days, respectively, only 36 and 38% of mice developed diabetes at 24 weeks of age. For Groups 3, 4 and 5, p<0.05 when compared to vehicle-treated group 1 via paired log-rank test following Kaplan Mayer analysis. Mice in Group 6 receiving 2 U of KLK1 every 7 days, 50% developed diabetes at 24 weeks of age. Mice in Group 2, treated with the lowest doses of KLK1 (0.08 U daily) developed diabetes at 69% at 24 weeks of age. Therefore, KLK1 protected against the development of T1D in female NOD mice, both in a dose dependent manner, and proportional to the frequency of administration. These results are also notable because KLK1 administration was started when the NOD mice were 6 weeks of age.

EdU Injections:

The method to detect DNA synthesis in proliferating cells was adopted from Salic et al., 2008. PNAS; 105(7):2415-20, which is based on the incorporation of 5-ethynyl-2′-deoxyuridine (EdU) into replicating DNA; and its subsequent detection by a fluorescent azide through a Cu(1)-catalyzed [3+2] cycloaddition reaction (“click” chemistry). In order to assess beta cell proliferation, random mice (at least 3 in each experimental group) were injected with EdU (Invitrogen) (25 mg/kg in 100 μl intraperitoneally×3 times a day) for 5 consecutive days during days 19-24 of KLK1 treatment. Experimental protocol for detection of the EdU label was modified from Ranall et al., 2010. BioTechniques; 48:379-386. Briefly, fresh-frozen in OCT compound pancreata of experimental animals were cryosectioned into 5 μm-thick sections; three tissue sections 150 μm apart from each other were placed on Superfrost glass slides; slides immersed for 5 minutes at −20° C. in fresh 100% acetone for tissue fixation, air-dried, washed 3×5 minutes in PBS, tissues circled with hydrophobic PAP pen, and incubated for 20 min at room temperature with staining mixture (10 μM; Cy5-labeled azide (Lumiprobe; #43030); 1 mM CuSO4; and 0.1M ascorbic acid in 100 mM Tris Buffer pH 8.5). Slides were then rinsed in TBS with 0.5% Triton X-100 for 15 min, washed 3×5 minutes in TBS; blocked with 1% BSA in PBS for 1 hr at room temperature, and processed for insulin staining. After completion of insulin staining, EdU incorporation into nuclei of proliferating cells was revealed under fluorescent microscope and images were acquired, recorded and analyzed. Overall, staining for EdU incorporation proved to be highly sensitive; and accomplishable in a short time. The small size of the fluorescent azides used for detection resulted in a high degree of specimen penetration. In contrast to BrdU, the EdU detection method does not require sample fixation or DNA denaturation, permits good structural preservation of the specimen, thus it was easy to combine with insulin staining in order to assess beta cell proliferation. Moreover, preservation of tissue integrity during EdU detection proved to be extremely important when analysis of the degree of insulitis was performed on the same sections.

Staining of Pancreatic Sections for Insulin, and Consequent Assessment of Insulin-Producing Mass:

Pancreatic sections were placed on slides, fixed, washed and blocked as described above. Tissue sections were then incubated with 100 μl/section of guinea pig anti-insulin primary antibody (AbCam; ab7842) diluted 1:100 diluted in 1% BSA overnight at 4° C. in a humid chamber; washed 3×5 minutes in PBS, incubated with 100 μl/section of TexasRed-labeled donkey anti-guinea pig secondary antibody (Jackson Immunoresearch; 706-075-148) diluted 1:100 in 1% BSA for 1 hr at room temperature in a humid chamber; wash 3×5 minutes in PBS; mounted in 3:1 Vectashield:DAPI (VectaShield Hardset; Vector Laboratories; H-1500); coversliped, and examined under the Nikon 90i fluorescent microscope within 2 days. Images were captured with a photometric CoolSNAP HQ2 camera, using the Nis-Elements AR software. Extensive morphometric analysis was then performed in blinded fashion on acquired images using Nis-Elements AR, and ImageJ software packages, in order to assess effects of KLK1 treatment on beta cell proliferation and insulin-producing mass.

Assessment of Beta Cell Proliferation:

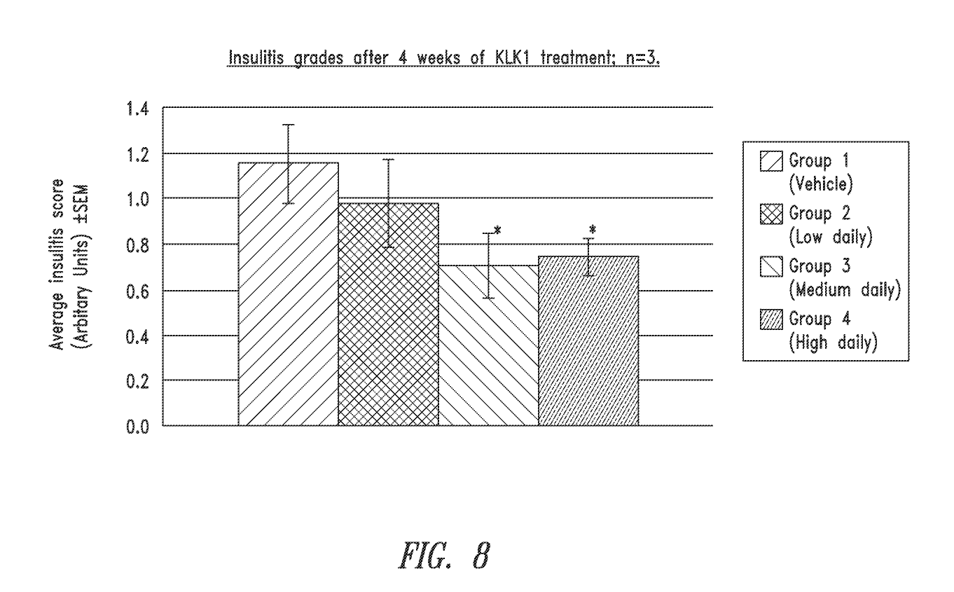

Effects of KLK1 treatment on beta cell proliferation were assessed in subgroups of experimental mice sacrificed at 10 weeks of age (4 weeks of KLK1 treatment) via detection of synthetic EdU nucleotide incorporation into the DNA of replicating cells, and counterstaining for insulin as described above. Cohort of three mice/group analyzed after 4 wks of KLK1 treatment, EdU treatment for last 5 consecutive days. Replicating beta cells were determined by fluorescent staining of pancreatic sections for insulin along with EdU. Cells double-positive for insulin and EdU were manually counted in blinded fashion in every islet detected. At least three slides per mouse (3 sections 150 microns apart per slide) were analyzed. Analysis of the effects of 4 weeks of KLK1 treatment on beta cell replication performed via detection of EdU incorporation into nuclei of insulin-positive beta cells. As summarized in FIG. 7, and contrary to previous reports that KLK1 induced beta cell proliferation, four weeks of continuous KLK1 treatment did not induce beta cell proliferation at any treatment dose. In fact, beta cell proliferation was significantly higher (p<0.05) for the negative control (vehicle-treated) animals vs. animals treated with high, medium of low KLK1 doses everyday (Analysis via 1-way Anova analysis with post-hoc Turkey multiple comparisons test). One possible explanation for a decrease in beta cell proliferation in KLK1 treated animals is protection of the beta cells from islet infiltration. Beta cells in the non-treated control NOD mice may replicate to replace beta cells damaged or destroyed by the autoimmune attack. Since the autoimmune attack is being attenuated in KLK1 treated NOD mice, the beta cells are not being destroyed or damaged by an autoimmune attack, there is no need for the beta cells to replicate. In another study wherein diabetes was induced in rats by administration of streptozotocin causing beta cell death, KLK1 treatment resulted in increased beta cells replication (data not shown). In this rat-streptozotocin study, KLK1 treatment stimulates beta cell replication to replace beta cells lost due to the streptozotocin. Further analysis was conducted to grade insulitis in the same NOD animals which were analyzed for beta cell proliferation.

Analysis and Grading of Insulitis:

Pancreatic sections from cohort of mice sacrificed at 4 weeks of KLK1 treatment were analyzed under fluorescent microscope for islet infiltration. Pancreata from three mice per group were analyzed in blinded fashion. Minimum of 50 islets per mouse were examined and graded for infiltration. The severity of insulitis was determined on the pancreatic sections fluorescently stained for insulin, as described above, and was scored in blinded fashion in accordance with the following grades: 0—intact islets, no infiltrating cells; 1—peri-insulitis, infiltrating leukocytes are located around islet mass, do not penetrate islet “capsule”; 2—insulitis, leukocytes clearly penetrating into islets, reducing beta cell zone by about 25%; 3—heavy insulitis, infiltrating leukocytes reduce beta cell mass by about 50%; 4—destructive “end-stage” insulitis, virtually no beta cells left within the islet infiltrate. Data in FIG. 8 is expressed as an average insulitis grade for indicated treatment group±S.E.M.

At ten weeks of age (study week 4), negative control (vehicle-treated) mice show active formation of insulitis (reflected in higher insulitis score); while mice treated with medium and high doses of KLK1 daily, have significantly lower insulitis scores (p>0.05 for groups 3 and 4 vs. group 1 via 1-way Anova analysis with post-hoc Turley multiple comparisons test), suggesting that KLK1 treatment delayed formation of insulitis (FIG. 8). The result that mice treated with KLK1 have lower insulitis scores corroborates the observation that beta cell proliferation was decreased in KLK1 treated mice; KLK1 treatment protects against an autoimmune reaction that destroys beta cells, and thus beta cell are not replicating to replenish destroyed cells. Of note, average insulitis scores for all groups of mice analyzed (FIG. 8) suggest that the majority of islets at this time-point are in the non-destructive insulitis stage. This observation is also consistent with the results in FIG. 2, that no animals have developed diabetes at 10 weeks of age.

Analysis of Insulin-Producing Masses on Cohort of Mice Sacrificed after 4 Weeks of Continuous Treatment with Different KLK1 Doses.

To determine pancreatic beta cell mass, pancreatic sections obtained from the cohort of three mice/treatment group sacrificed after 4 weeks of KLK1 treatment. Sections were fluorescently stained for insulin; and beta cell masses were calculated by measuring insulin-positive stained area of each islet on the section, determining summarized insulin-positive stained area for each section, which was then divided by the total pancreas area of the section, and resultant value multiplied by the total pancreas weight. Three slides per mouse were analyzed in blinded fashion, with the sections being at least 150 microns apart. No statistically significant differences were found in beta cell masses in animals from KLK1 treatment groups analyzed (data not shown). Although vehicle-treated animals have the highest degree of insulitis (FIG. 8), this observation does not translate into reduced beta cell mass. This observation confirms the previous observation that at 10 weeks of mouse age, (study week 4), no destructive insulitis is present in islets of mice from all treatment groups.

Summarizing the analysis of beta cell proliferation, insulitis formation, and assessment of insulin-producing mass, at 4 weeks of continuous KLK1 treatment resulted in the significant attenuation of insulitis development in animals receiving high and medium doses of KLK1. Additionally, treatment with KLK1 appears to protect the beta cells from the autoimmune reaction as evidenced by the attenuated insulitis development. Animals treated with high and medium doses of KLK1 daily, had significantly lower numbers of proliferating beta cells, which as described above also indicates protection of beta cells, compared to vehicle-treated controls. Thus, 4 weeks of KLK1 treatment mostly resulted in immunomodulatory mode, attenuating formation of insulitis.

To assess if immunomodulatory effects observed at 4 weeks of KLK1 treatment continue throughout the whole 18 week experimental treatment regimen, beta cell masses, and insulitis levels were determined in all remaining non-diabetic mice at the end of 18 weeks of KLK1 treatment. Pancreatic sections were analyzed under fluorescent microscope for islet infiltration. Data expressed as an average insulitis grade for indicated treatment group±S.E.M. (color-coded)-number of animals analyzed in each group in blinded fashion. Minimum of 80 islets per mouse were examined and graded for infiltration. As summarized in FIG. 9, 18 weeks of KLK1 treatment significantly reduced the degree of insulitis in medium dose daily, and in high doses daily and 3× a week, compared to the rest of treatment regimens (p<0.05 vs. Groups 1, 2 and 6 via 1-way Anova analysis with post-hoc Turkey multiple comparisons test). Since development of spontaneous disease in NOD mice depends on progression of insulitis into diabetes, the observed reduction in insulitis formation in animals treated with daily high and medium KLK1 doses, or 3× a week high dose of KLK1, correlates with protection from the development of spontaneous diabetes (illustrated in FIG. 2).

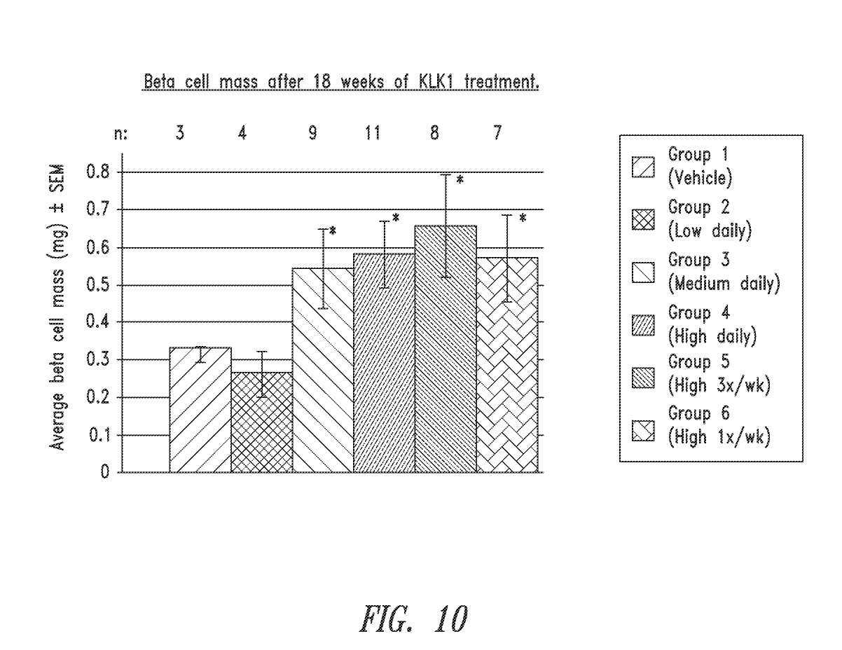

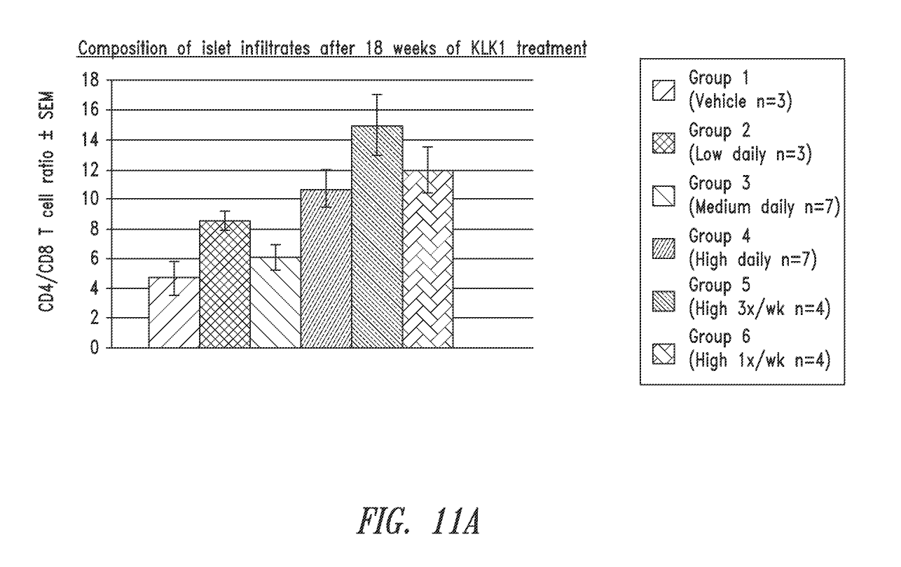

Beta cell mass was also analyzed in mice after 18 weeks. Pancreatic sections obtained from all non-diabetic mice were fluorescently stained for insulin; and beta cell masses were calculated as described above. Three slides per mouse were analyzed in blinded fashion with the sections being at least 150 microns apart. n—number of animals in each group (color-coded). As summarized in FIG. 10, analysis of beta cell masses of animals from all treatment groups demonstrated that KLK1 treatment resulted in significant protection of insulin-producing cells in groups treated with high and medium dose of KLK1 daily, and with high doses of KLK1 daily, 3× a week, and 1× a week, compared to control animals and animals treated with low KLK1 dose daily (p<0.05 vs. Groups 1 and 2 via 1-way Anova analysis with post-hoc Turkey multiple comparisons test).

Overall, at the end of 18 weeks of continuous treatment, KLK1 administered at the medium and high doses daily, and 3× a week, resulted in a significant reduction of insulitis formation (FIG. 9); and consequently protected beta cells (beta cell mass) (see FIG. 10) from destruction by cytotoxic lymphocytes. To elaborate on these findings, an analysis of insulitis composition was performed on the pancreatic sections obtained from all animals which remained non-diabetic mice at the end of 18 weeks of KLK1 treatment.

Analysis of Islet Infiltrates Composition:

Assessment of islet infiltrates' composition after 18 weeks of KLK1 treatment was done by immunofluorescent staining of pancreatic sections for CD4 and CD8 surface markers of T helper and cytotoxic T lymphocytes, respectively. Briefly, slides containing pancreatic sections were prepared, fixed, washed and circled with hydrophobic PAP pen as described in analysis of beta cell proliferation section, were blocked with 100 μl/section of 1% donkey serum in PBS for 30 minutes at room temperature in a humid chamber, then incubated with 100 μl/section of primary rat anti-mouse CD4 antibody (BioLegend; #100401) diluted 1:100 in 1% donkey serum for 1 hr at room temperature in a humid chamber; washed 3×5 minutes in PBS; incubated with 100 μl/section of secondary donkey anti-rat, Cy5 fluorophore-labeled antibody (Jackson Immunoresearch #712-175-153) diluted 1:100 in 1% donkey serum for 1 hr at room temperature in a humid chamber; washed 3×5 minutes in PBS; blocked with 100 μl/section of 1% BSA in PBS for 30 minutes at room temperature; then incubated with directly FITC-labeled rat anti-mouse CD8 antibody (BioLegend #100726) diluted 1:100 in 1% BSA for 1 hr at room temperature, washed 3×5 minutes in PBS; mounted in 3:1 Vectashield:DAPI (VectaShield Hardset; Vector Laboratories; H-1500); cover-slipped, and examined under the Nikon 90i fluorescent microscope within 2 days. Images were captured a photometric CoolSNAP HQ2 camera, using the Nis-Elements AR software. Image analysis was completed in blinded fashion using Nis-Elements AR and Image) software packages.

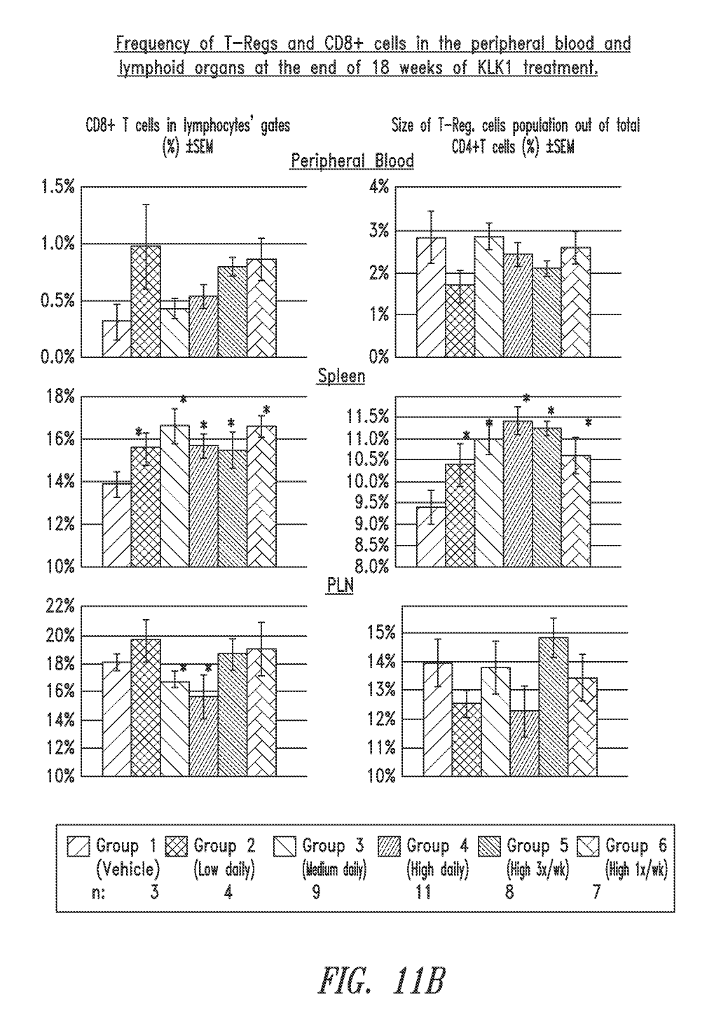

Pancreatic sections from all non-diabetic mice at the end of 18 weeks of KLK1 treatment were analyzed under fluorescent microscope for islet infiltrates composition after immunofluorescent staining for CD4 and CD8 T cell markers. Numbers of CD4 and CD8 positive cells in each individual islet were manually counted in blinded fashion. Data expressed as an average ratios of CD4+ cells to CD8+ cells for indicated treatment group±S.E.M. n—number of animals analyzed in each group. Analysis of islet infiltrates composition is summarized in FIG. 11A. Mice in all experimental groups treated with high concentrations of KLK1 (1× daily, 3× week, and 1× week) had significantly elevated ratios of CD4+ to CD8+ T cells found in pancreatic islets, when compared to animals from vehicle, and low dose everyday KLK1-treated groups (p<0.05 vs. Groups 1, 2 and 3 via 1-way Anova analysis with post-hoc Turkey multiple comparisons test). Detailed analysis of infiltrate composition revealed that observed elevation of CD4+ to CD8+ T cell ratios in high dose of KLK1-treated groups was associated with the reduction of numbers of CD8+ cytotoxic T cells, found in islet infiltrates; while absolute numbers of CD4+ helper T cells in infiltrates were not significantly affected by KLK1 treatment. Thus, treatment with high doses of KLK1 over 18 week period significantly altered overall composition of pancreatic infiltrates, largely reducing population of infiltrate-bound CD8+ cytotoxic T cells and increasing the ratio of CD4+ helper T cells in the total T cell population in islets. Such transformation of infiltrate composition is generally regarded to provide for diminished aggressiveness of diabetogenic autoimmune attack, as CD8+ cytotoxic T cells are the major effectors, delivering direct cytotoxic elimination of beta cells.

Effects of Recombinant Human KLK1 Treatment on Fasting Blood Glucose Levels.

Fasting blood glucose levels were determined in 4 to 8 mice selected from each treatment groups, and the results are depicted in FIGS. 3A to 3F. Fasting blood glucose levels were determined in the mice by removing food for 12 hours prior to testing (water provided ad libitum). Fasting blood glucose levels were determined in the mice twice a week until the end of the 18 week experiment, or until the mouse developed diabetes (non-fasting blood glucose levels were higher than 250 mg/dL for two consecutive days). Most mice demonstrated normal or near normal fasting blood glucose levels (60-130 mg/dL). These results confirm the above observation that NOD mice treated with recombinant human KLK1 did not develop diabetes. Additionally, at all doses, KLK1 treated pre-diabetic NOD mice exhibited euglycaemia during fasting and did not result in fasting hypoglycaemia, suggesting that KLK1 treatment did not adversely affect beta cells function resulting in hyperinsulinemia.

Glucose Tolerance Tests on NOD Mice Treated with Recombinant Human KLK1.

Intraperitoneal glucose tolerance tests (IPGTT) were administered to animals on treatment day 97 (week 14), day 112 (week 16) and day 125 (week 18). For this, glucose (2.5 g/kg) was injected i.p. into 12 hour fasting animals, and measurements of blood glucose levels was performed at 0, 30, 60, 120 and 180 minutes after glucose injection. An IPGTT performed in non-diabetic mice or animals with normal functioning beta cells, the IP injection of glucose will be absorbed into the bloodstream and result in an increase in blood glucose levels, which will trigger an increase in insulin release from beta cells. Non-diabetic animals may have a moderate increase in blood glucose levels from fasting levels to about 250 mg/dL approximately 15 minutes after glucose injection, and a return to near normal blood sugar levels by 60 minutes post injection.

As depicted in FIG. 4A, vehicle treated (negative control) NOD mice on day 97 (treatment week 14), IPGTT resulted in an abnormally high increase in blood glucose levels (greater than 300 mg/Dl) and a slow decrease over time. The blood glucose levels did not return to fasting levels even at 120 minutes after glucose injection. This poor response to the IPGTT was also evident at days 112 and 125 (treatment week 16 and 18, respectively). The NOD mice on day 97 were 20 weeks old, and typical age of onset for type 1 diabetes is 18 to 20 weeks of age. Pre-diabetic NOD mice, at this age range, are known to have impaired beta cell function, likely resulting from insulitis and decreased beta cell mass described above, and a poor insulin release response to glucose challenge prior to the onset of type 1 diabetes. As such, the high blood glucose peak and slow decline in blood glucose levels in the IPGTT results for vehicle treated NOD mice demonstrate impairment of beta cell function.

IPGTT results for mice treated with low doses of KLK1 (FIG. 4B) also demonstrated a high blood glucose peak and a slow decline in blood glucose levels, similar in pattern to the vehicle (negative control) mice, suggesting low doses of KLK1 did not protect beta cell function against becoming impaired. Mice treated with the medium daily dose of KLK1 (FIG. 4C), at day 97 (treatment week 14) and day 112 (treatment week 16) had higher peak blood glucose levels than expected for non-diabetic mice, but the blood glucose levels decreased to near normal levels by 60 minutes. However, by day 125 (treatment week 18), the peak glucose levels were higher and the rate at which glucose levels returned to normal were longer, suggesting medium doses of KLK1 provided some protection to beta cells, and confirming above observations of decreased insulitis and preservation of beta cell mass with increased KLK1 dose.

Animals treated with the high daily dose of KLK1 at day 97 and 112 (treatment week 14 and 16, respectively) had moderate peak glucose levels (less than 250 mg/Dl) at 15 minutes post glucose injection and a rapid reduction to normal blood glucose levels by 60 minutes (see FIG. 4D). Such an IPGTT is considered normal and suggests normal functioning of beta cells, which is consistent with the decreased insulitis and preservation of beta cell mass at this KLK1 dose. By day 125 (treatment week 18), the animals had an increase in peak blood glucose levels, though levels decreased to normal levels within 60 minutes. Mice treated with high levels every 3 days (FIG. 4E) or every 7 days (FIG. 4F) had IPGTT results intermediate between the medium daily and high daily dose animals, indicative that perhaps KLK1 treatment at these frequencies was not sufficient to fully protect the beta cells from autoimmune attack.

KLK1 treatment restored IPGTT results in NOD mice in a dose dependent and in a frequency dependant manner. At lower doses or less frequent doses, KLK1 treatment appears to have a small ameliorating effect on beta cell impairment associated with T1D since the IPGTT indicated the NOD mice had impaired beta cell function, but not to the extent of untreated mice. At higher KLK1 doses, mice have normal or near normal responses to IPGTT, indicating KLK1 treatment has ameliorated beta cell impairment associated with T1D.

Measurements of Insulin, C-Peptide and TGF-Beta Levels in Serum:

100 μl of blood was withdrawn from the retro-orbital venous sinus of all non-diabetic at the time of blood withdrawal animals, at treatment days 0, 52, 74, and 107. At the end of treatment (days 125-129), blood was withdrawn directly from the hearts of animals immediately after sacrification. Blood was processed to serum by centrifugation, aliquoted, and sera from earlier time-points were stored at −80° C., till the latest time-point samples become available; then all samples were analyzed using ELISA-based kits for the determination of mouse insulin and TGF-beta: (Millipore cat #EZRMI-13K and R&D cat #SMB100B; respectively), according to the manufacturer's instructions. Unfortunately, serum aliquots obtained on day 107 were accidentally defrosted, making further analysis of samples from this time-point unavailable.

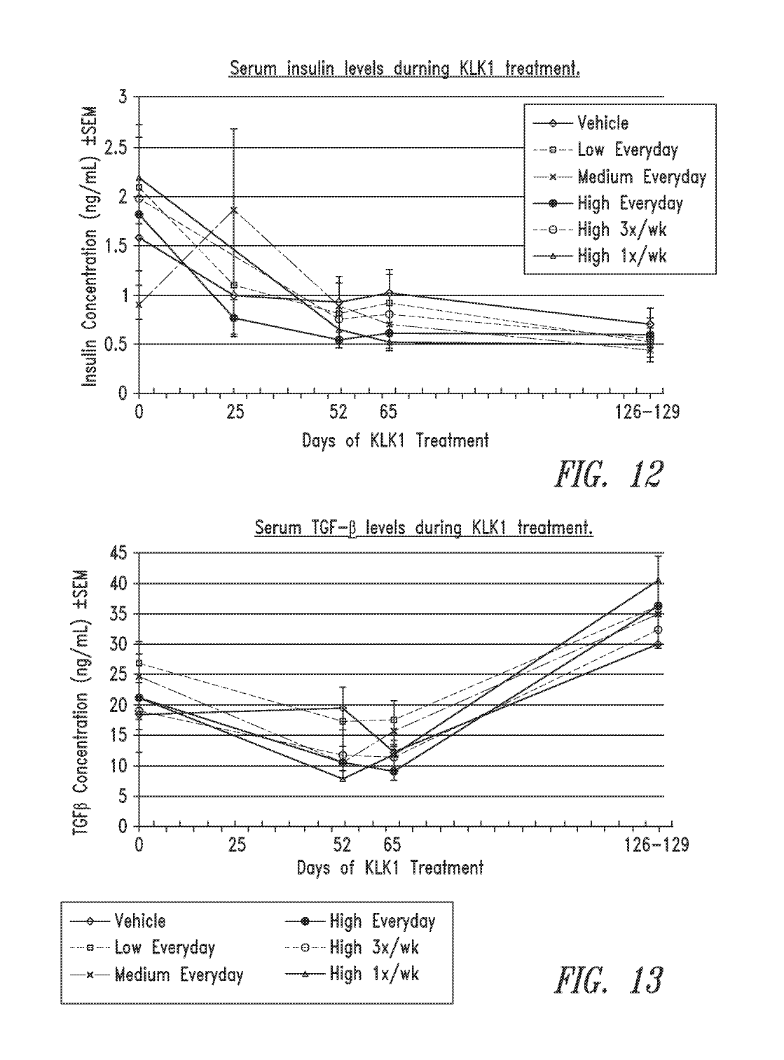

Serum insulin concentrations in the experimental animals were not significantly affected by KLK1 treatment at any time-point tested. There was no difference in serum insulin levels between the non-diabetic animals in the negative control group, and in any of the KLK1 treatment groups (see FIG. 12). Overall tendency for slight decrease in insulin concentrations with the progression of time was observed for all experimental groups of mice independent of KLK1 treatment (see FIG. 12). As the animals were not diabetic when the sera was isolated, they would be expected to have detectable circulating insulin present. Importantly, KLK1 treatment of NOD mice does not appear to result is hyperinsulinemia, which would be dangerous.

Since KLK1 treatment of NOD mice resulted in increase CD4+/CD25+/Foxp3+ Tregs, and since TGF beta is believed to be important in the differentiation of T cells into Tregs, the circulating levels of TGF beta were measured to determine if levels increased in response to KLK1 treatment. No significant differences of serum TGF-β concentrations were observed in groups of experimental animals throughout KLK1 treatment. Overall tendency of slight increase of TGF-β concentrations in the peripheral blood at the end-of-experiment time point was detected in all groups of animals, independently of KLK1 treatment (see FIG. 13). This result may be explained because TGF-β is a potent cytokine; it is widely known to be produced in discrete amounts and act locally, within the site of production. Biologically active TGF beta has a very short half-life in circulation (<5 minutes), and thus, systemic levels of TGF-β are rarely affected by the local increase of this cytokine concentration.

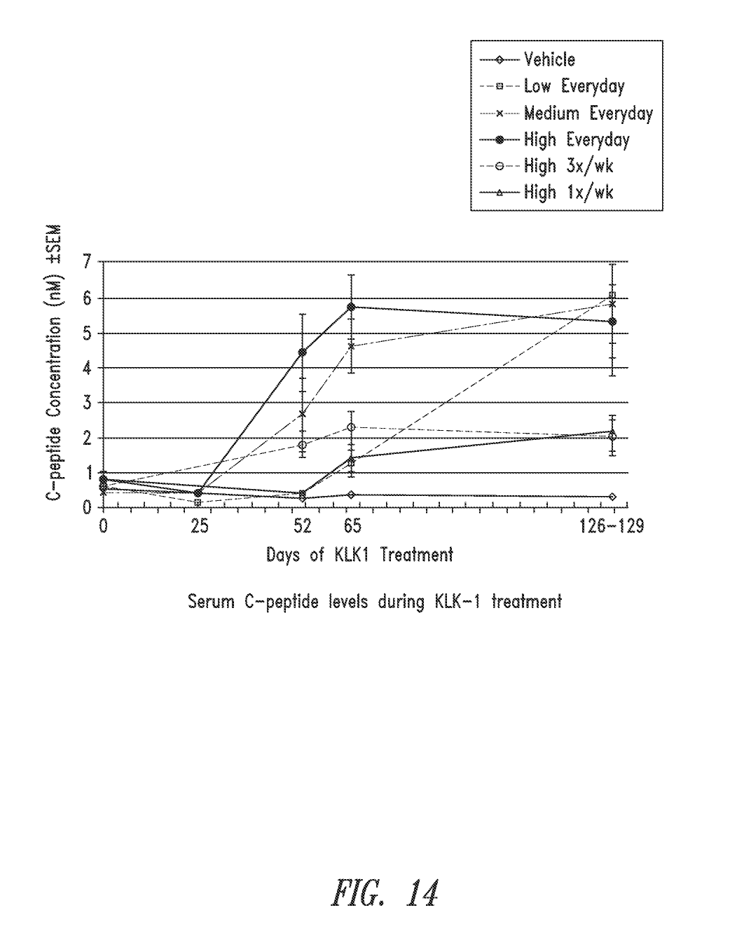

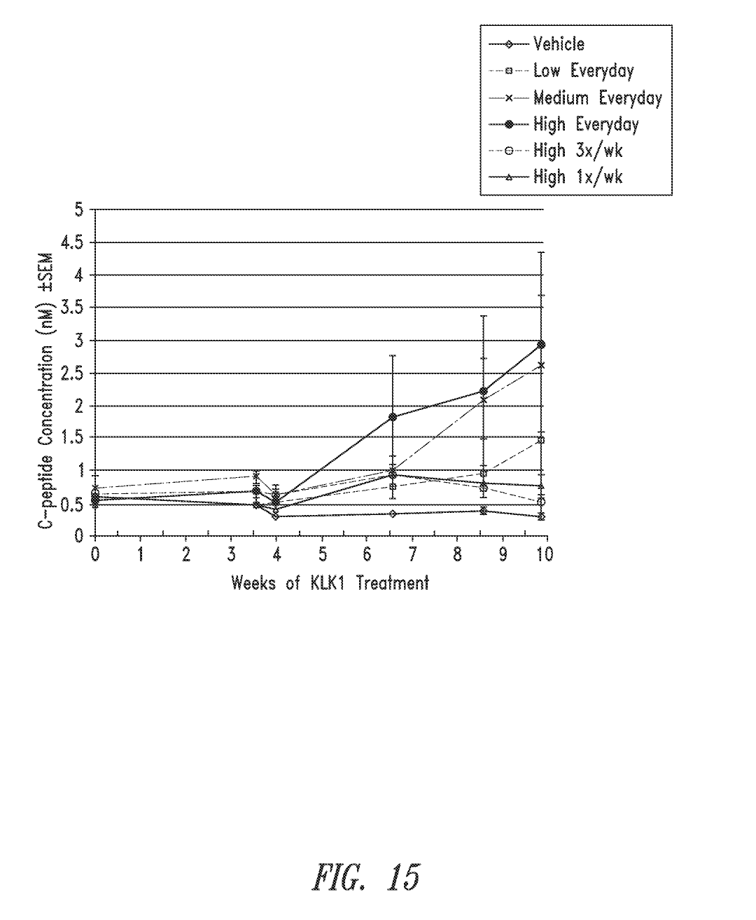

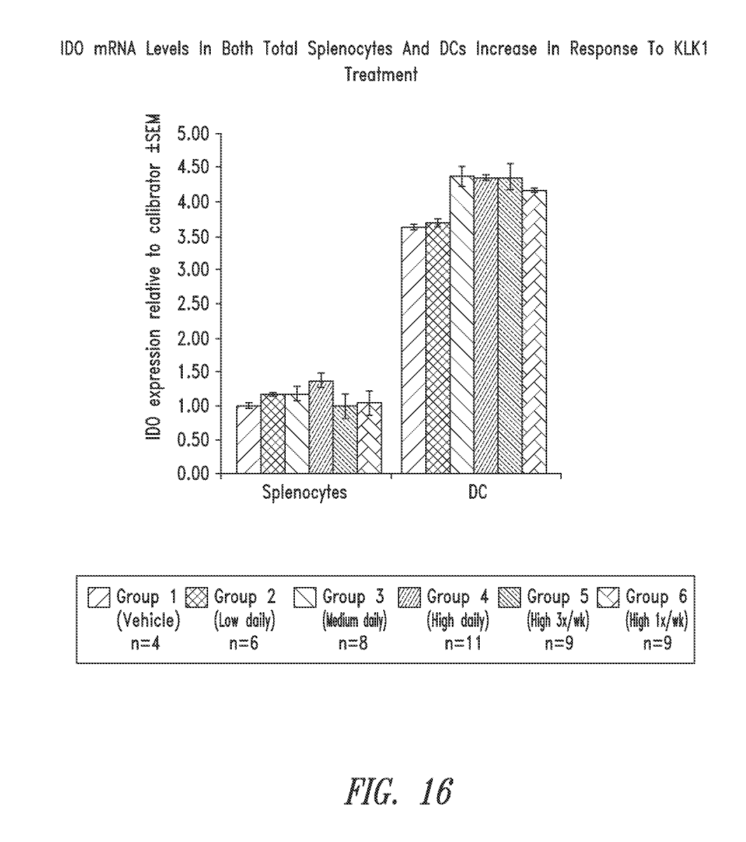

The above noted serum samples were also analyzed for mouse C-peptide using ELISA-based kit (ALPCO cat #80-CPTMS-E01), according to the manufacturer's instructions. As evident from FIG. 14, continuous KLK1 treatment had a profound time-delayed effect on C-peptide concentrations, measured in peripheral blood of experimental animals. Low amounts of serum C-peptide were detected in all groups of animals at the beginning of KLK1 treatment, and remained unchanged during the initial period of at least 25 days at approx. 0.5 nM, which is in the C-peptide concentration range for a non-diabetic mouse. The C-peptide levels then started to increase by statistically significant amounts in a KLK1 dose- and frequency-of-treatment-dependent manner. By day 52 of KLK1 treatment, the C-peptide levels in mice treated with high daily KLK1 had increased to approx. 9 fold to 4.5 nM, in animals receiving medium daily dose of KLK1, C-peptide concentration increased 5 fold to approx. 2.5 nM, and high 3× per week resulted in a 4 fold increase to approx. 2 nM C-peptide (FIG. 14). In animals receiving low KLK1 dose daily, and high KLK1 dose once-a-week, serum C-peptides levels had not increased after 52 days of treatment. By the end of the experiment (days 126-129), the serum C-peptide concentrations were significantly higher in all KLK1 treatment groups vs. negative control group. Also, at this final time-point, higher overall serum C-peptide levels were present based on the dose and frequency of KLK1 treatment. Specifically, in experimental groups receiving daily KLK1 treatment (high, medium, and low doses) the average serum C-peptide concentrations were approx. 4 to 7 nM, which was about 8-14 fold higher than control vehicle treated animals or levels at the start of the experiment. In groups receiving KLK1 less frequently (1× a week and 3× a week), approx. 2 nM or about 4-fold higher levels of C-peptide were detected compared to vehicle-treated group, which maintained unchanged at approx. 0.5 nM concentrations of C-peptide throughout the experiment. To determine if the above results are reproducible, another group of NOD mice were treated with KLK1 during a 10-week treatment regimen and their C-peptide levels measured. As observed in the 18 week treatment regimen, the C-peptide levels in mice in the 10 week treatment regimen increased in proportion to the dose and dose frequency of KLK1 (FIG. 15), demonstrating the increase in C-peptide levels is reproducible. The C-peptide levels in this 10 week experiment increased to approx. half the levels observed in the 18 week study. The lower C-peptide levels may be due to the shortened KLK1 treatment time as it appears C-peptide levels had not plateaued and were still increasing at the end of the 10 week experiment.

Although C-peptide and insulin are released by beta cells into the bloodstream in equimolar proportions, C-peptide has a longer half-life in peripheral tissues than insulin and C-peptide is therefore detected at higher molar concentrations in circulation. The apparent increase in C-peptide with KLK1 treatment would be predicted to have also resulted in increased insulin concentrations detected in circulation and/or hypoglycemia. However, unlike the C-peptide concentrations, serum insulin concentrations in the experimental animals were not affected by KLK1 treatment at any time-point tested (FIG. 12) and hypoglycemia was not observed in any of the animals (FIGS. 3A to 3F). Overall there was a slight decrease in insulin concentrations for all experimental groups of mice as the experiment progressed, independent of KLK1 treatment.

One possible explanation for the increased C-peptide levels is an insulinoma. However, there was no increase in insulin levels, no increase in beta cell mass observed in any KLK1 treated animal, no indication of tumors on histological examination, and most importantly, no hypoglycemia was detected in any animal. KLK1 may act as a C-peptide preserving agent, negatively regulating C-peptide catabolism. This is supported by the gradual C-peptide increase, with no change (actual decrease) in insulin levels. The kidney is the main site for C-peptide catabolism and excretion. Given that the main function of KLK1 is to produce biologically active kinins, which act through bradykinin receptors located in the kidney, the increased bradykinin receptor signaling in the kidney might affect C-peptide kidney reabsorption, catabolism, and urinary excretion. KLK1 may also inhibit an enzyme associated with degradation of C-peptide.

The discrepancy between the effects of KLK1 treatment increasing serum C-peptide concentrations, suggesting increased insulin secretion, and absence of detection of increased insulin levels may be explained by the low half-life of insulin molecules in peripheral blood of NOD mice (less than 3 minutes), due to immediate insulin utilization by peripheral tissues. Much more stable C-peptide molecules have a half-life in peripheral blood is at least 10 times longer, compared to insulin (reviewed by Luppi et al., Pediatric Diabete.; 12:276-92, 2011), and thus provide better target for detection in peripheral blood. The observed increase in C-peptide levels in response to KLK1 treatment may be partially explained by the samples being taken from non-fasting animals. Additionally, insulin levels are usually tightly controlled in peripheral blood, increasing in response to an increase of blood sugar levels following a meal, and then rapidly falling back to baseline after a meal to avoid hypoglycemia. Finally, NOD mice are known to become insulin resistant, especially if the autoimmune disease leading to T1D is suppressed. The increase in insulin secretion with KLK1 treatment, as evidenced by C-peptide levels, may be the result of normally occurring insulin resistance in NOD mice, and have been counteracted by increasing insulin turnover to avoid detection of hyperinsulinemia or hypoglycemia.

The observed increased in C-peptide levels with KLK1 therapy may be due to an inhibition in breakdown of C-peptide, which may have additional therapeutic potential. Rather than being an inert byproduct of insulin biosynthesis, C-peptide has recently been demonstrated to bind receptors at the cell surface and activate signal transduction pathways. Type 1 diabetic patients undergoing insulin replacement therapy are injected with insulin but not C-peptide. This lack of C-peptide has been implicated as a cause for many of the complications associated with Type 1 diabetes, especially neuropathy but also nephropathy and retinopathy (Wahren J, Ekberg K, Johansson J, et al., Role of C-peptide in human physiology. Am J Physiol Endocrinol Metab 278(5):E759-68, 2000).

In several clinical trials, treatment of T1D patients with C-peptide improved sensory nerve dysfunction and structural abnormalities, specifically increasing sensory nerve conduction velocity, vibration perception, regression of nodal changes, increased axonal regeneration, and improved autonomic nerve function (heart rate variability). Treatment with KLK1 would increase C-peptide levels, and have a therapeutic effect on diabetic neuropathy, specifically:
Peripheral neuropathy, the most common type of diabetic neuropathy, causes pain or loss of feeling in the toes, feet, legs, hands, and arms.
Autonomic neuropathy causes changes in digestion, bowel and bladder function, sexual response (erectile dysfunction in men and vaginal dryness in women), and perspiration. It can also affect the nerves that serve the heart and control blood pressure, as well as nerves in the lungs and eyes. Autonomic neuropathy can also cause hypoglycemia unawareness, a condition in which people no longer experience the warning symptoms of low blood glucose levels.
Proximal neuropathy causes pain in the thighs, hips, or buttocks and leads to weakness in the legs.
Focal neuropathy results in the sudden weakness of one nerve or a group of nerves, causing muscle weakness or pain. Any nerve in the body can be affected.

KLK1 treatment resulting in increased C-peptide levels may improved renal function (normalized glomerular filtration, decreased albumin excretion) and reduce diabetes-induced structural changes (decreased mesangial expansion). KLK1 treatment resulting in increased C-peptide levels may also result in increased regional blood flow to the muscle, myocardium, nerve and kidney. Therefore, KLK1 treatment resulting in improved or restored C-peptide secretion in type 1 diabetic patients would have significant benefit in preventing or alleviating the long term complications associated with Type 1 diabetes, especially neuropathy but also nephropathy and retinopathy.

The observed increase in C-peptide levels associated with KLK1 treatment may also serve as a biomarker to demonstrate KLK1 treatment is having an effect in a T1D patient. Specifically, an increase in C-peptide levels following KLK1 treatment in a T1D patient (e.g., (recent onset, established, LADA) may be used to dose patients, and KLK1 dosing (e.g., dosage frequency and/or dosage amounts) may be increased until an increase in C-peptide levels is observed, compared to C-peptides levels prior to KLK1 treatment. Alternatively, C-peptide levels may also be monitored during KLK1 treatment of increased risk patients of developing type 1 diabetes. KLK1 may be administered to increased risk patients until C-peptide levels increase, or the KLK1 dosing may be increased until C-peptide levels increase such that the C-peptide levels fall within a desired range.

Normal C-peptide levels for a fasting test are generally considered to include any result between about 0.5 ng/ml and about 3 ng/ml; however, even healthy subjects without diabetes may occasionally have levels outside of this range. Certain exemplary C-peptide range values for normal or healthy subjects include the following: children (<about 15 years old) 8:00 a.m. fasting—0.4 to 2.2 ng/ml; adults 8:00 a.m. fasting—0.4 to 2.1 ng/ml; two hours after a meal—1.2 to 3.4 ng/ml; and two hours post glucose load—2.0 to 4.5 ng/ml. Hence, certain embodiments include increasing the KLK1 dosage (frequency and/or amount) until C-peptide levels of a given subject fall within one of the above ranges, as desired.

Treatment with Recombinant Human KLK1 does not Significantly Alter Blood Pressure in Pre-Diabetic NOD Mice.

KLK1 release the vasoactive peptide, Lys-bradykinin, from low molecular weight kininogen. As such, administration of KLK1 may result in generation of high levels of bradykinin within the animals, resulting in a drop in blood pressure. To determine if recombinant human KLK1 had an effect on the blood pressures of the mice, blood pressure and heart rate measurements were taken from 5 random mice, using blood pressure monitoring system (IITC Life Science Inc.), which obtains and records systolic, diastolic, and mean pressure and heart rate utilizing photoelectric sensor detection of blood pressure pulses. This is a non-invasive, minimal-disturbance tail-cuff based technique. FIG. 5 depicts the effect of KLK1 treatment on the average systolic blood pressure in mice at treatment day 77, treatment day 98, and treatment day 126. Treatment of NOD mice with KLK1 did not have a significant effect on blood pressure and heart rates throughout the 18 week treatment program and specifically, no decrease in systolic, diastolic, and mean blood pressure.

Recombinant Human KLK1 Administration Results in Increased CD8+ T Cells and Treg Cells (CD4+CD25+Foxp3+) in Spleens of NOD Mice.

KLK1 properties in attenuating diabetic auto-immune processes were investigated by analyzing the numbers of T cells with regulatory phenotype in the blood, spleens and pancreatic lymph nodes (PLNs), and correlating the observed findings with changes in beta cell function as evidenced in the IPGTT's.

At the end of 18 weeks of KLK1 treatment, all remaining mice (animals were 24 weeks of age at this point) were sacrificed. The pancreata, spleens, and PLNs were collected from the 24 week old mice and from mice sacrificed at any prior time-point due to diabetes onset. Numbers of T cell with regulatory phenotype in spleens and PLNs were assessed via FACS analysis (CellQuest™ software, Guava™ EasyCyte 8HT™ multi-laser cell analyzer, Millipore) after isolation of splenic and lymph-node lymphocytes and their staining with Foxp3, CD4 and CD25 (BD Pharmingen™) fluorolabeled antibodies. Suppressor T cells are a subset of CD4+ cells and are also known as CD25+/Foxp3+ regulatory T cells (Tregs). In animal studies, Tregs that express Foxp3 appear to have an important function in immune tolerance, especially self-tolerance. Decreased numbers of Foxp3 positive regulatory T cells are found in a number of autoimmune diseases, including type 1 diabetes.

Analysis of CD8+ Cells Frequencies in Peripheral Lymphoid Organs.

Numbers of CD8+ T cells within peripheral blood, spleens and PLN were assessed via FACS analysis after isolation of peripheral blood, splenic and lymph-node leukocytes and their staining with a CD8 fluorolabeled antibody. Briefly, samples were processed in for analysis of T regulatory cells frequencies in peripheral lymphoid organs to the blocking step. Then, directly-labeled anti-CD8-FITC (BioLegend; #100723) antibody was added to the cells and incubated for 30 minutes on ice. The CD8 antibody was diluted 1:200 in FACS buffer. The cells were then spun down at 1400 rpm for 5 minutes at 4° C. then washed with 200 μL FACS buffer, repeated twice. Cells were re-suspended in 200 μL FACS buffer and then analyzed.

Cells isolated from spleens, blood and PLNs of mice were analyzed by fluorescence activated cell sorting (FACS) to determine the percentage of CD4+ cells that were CD25+/Foxp3+ in the various KLK1 treatment groups. In cells isolated from PLN and peripheral blood samples, FACS analysis determined there were no differences in the percentage of CD4+ cells that were CD25+/Foxp3+ between the vehicle control group and the various KLK1 treatment groups (FIG. 11B, right side graphs). However, FACS analysis of cells from the spleen revealed a clear increase in Tregs between the vehicle control group and KLK1 treatment groups, and the percentage of Tregs increased with increased KLK1 dose (FIG. 6, FIG. 11B). Mice in group 4, treated with the highest daily dose of KLK1, had statistically significant (p<0.01) higher percentage of Foxp3+ cells compared to vehicle treated mice. Also, a trend was observed with higher doses of KLK1 and increased frequency of KLK1 doses resulting in a higher percent of Foxp3+ cells. This observation suggests that KLK1 treatment prevents autoimmune destruction of beta cells in NOD mice, which appears to be associated with increased numbers of regulatory T cells (CD4+ cells that were CD25+/Foxp3+).

Frequency of CD8+ T cells, detected in peripheral blood of non-diabetic by the end of experiment animals, remained unaffected by 18 weeks of KLK1 treatment (FIG. 11B, Left side graphs). However, KLK1 treatment resulted in opposite effects on numbers of CD8+ cells in the spleens and pancreatic lymph-nodes of experimental mice. While significantly higher percentages of CD8+ T cells were found to reside in spleens of all groups of animals treated with KLK1, compared to vehicle-treated group; in the PLNs, treatment with daily medium and high doses of KLK1 induced significant reduction in the percentages of CD8+ cells, vs. vehicle-treated controls (FIG. 11B, left side graphs). Summarizing these findings, KLK1 treatment affected distribution of CD8+ cytotoxic T cells in lymphoid organs.

During pathogenesis of T1D, pancreatic lymph-nodes (PLN) are the major site of antigenic priming and consequent proliferation of diabetogenic CD8+ cytotoxic T cells. Lower numbers of CD8+ T cells found in PLNs of animals treated with medium and high doses of KLK1 daily, thus, may correspond to the less aggressive diabetogenic process, and reflect the mechanism of the protective effect KLK1 treatment had on the development of spontaneous diabetes. This suggestion is supported by the results of the analysis of islet infiltrates (FIG. 11A), which show that KLK1 treatment resulted in the reduction of aggressiveness of insulitis, as numbers of CD8+ cytotoxic T cells were significantly lower in pancreatic infiltrates of mice treated with high doses of KLK1. In agreement, in the spleen, which as the largest secondary lymphoid organ contains a significant population of naïve CD8+ T cells, numbers of CD8+ T cells were slightly increased following KLK1 treatment.

Overall, an autoimmune mechanism driving development of T1D depends on the dynamic migration of CD8+ cytotoxic T cells into pancreas-draining regional lymph-nodes (PLN) and then into the target tissue of pancreatic islets. KLK1 treatment resulted in the redistribution of CD8+ cytotoxic T cells within lymphoid and target tissues, significantly reducing numbers of cytotoxic T cells in pancreatic lymph-nodes and islets. Such redistribution most likely is the consequence of an altered migration of CD8+ cytotoxic T cells into both, PLN and pancreatic islets. Due to the known proteolytic properties of KLK1, it might act via direct proteolysis of any of CD8+ cytotoxic T cells surface molecules, which are involved in the migration of CD8+ T cells into both, PLN and pancreatic islets. Alternatively, KLK1 can affect CD8+ cytotoxic T cells homing indirectly, via proteolytic modification of endothelial and/or systemic (chemokines; cytokines, etc) factors implicated in the regulation of T cell migration.

### Example 4

The data in these experiments demonstrate that KLK1 treatment in NOD mice is associated with increased expression of IDO in dendritic cells (DC's). Indoleamine-pyrrole 2,3-dioxygenase (IDO or INDO EC 1.13.11.52) or “IDO” is an immunomodulatory enzyme produced by certain alternatively activated macrophages and other immunoregulatory cells. The increase in IDO expression could thus be used a biomarker to determine the effectiveness of KLK1 dose or treatment in patients.

Preparation of Mouse Splenocytes.

NOD mice were treated with KLK1 polypeptides, as described in Example 3. Single-cell suspensions from approximately 70% of a mouse spleen were prepared after mechanical disruption of the spleen in 3-4 mL PBS using 3 mL syringe plunger. The disrupted spleen was transferred with 10 mL of PBS into 15 mL tube and further mechanically disrupted by extensive pipetting. After short incubation (1-2 min) at room temperature to allow stromal elements to settle, the single cells in the supernatant were transferred to new tubes. The collected cells were then pre-filtered through 30 μM mesh and pelleted by centrifugation at 400 g for 5 min. Red blood cells were then lyzed with LCK Lysing buffer (Lonza; cat.#10-548E) at room temperature for 5 minutes followed by addition of 10 ml PBS to stop the reaction. Cells were filtered again through 30 μM mesh, washed once with PBS and the final splenocyte pellet was resuspended in 800 μL PBS containing 0.5% FBS and 2 mM EDTA. A 40 μL aliquot was taken in new tube containing 1 mL TRIzol reagent (Invitrogen; cat #15596018) and then frozen at −80° C.

Isolation of Dendritic Cells (DCs).

DCs were isolated from total splenocytes using CD11c Microbeads (Miltenyi Biotec GmbH; cat #130-052-001) following standard manufacturers protocol. Briefly, splenocytes were pooled from 2-3 mice of the same experimental group. Splenocyte suspensions were brought to the total volume of 1 ml, then blocked with 16 μL FC Receptor blocker (eBioscience; cat #14-0161-85) for 5 minutes at 4° C., and then incubated for 20 minutes at 4° C. with 200 μL anti-CD11c beads. The magnetically labeled CD11c+ cells were purified by purification through two MS columns (Miltenyi Biotec GmbH; cat #130-042-201). During purification columns were washed three times with 500 μL PBS containing 0.5% FBS and 2 mM EDTA and cells were eluted with 1 mL buffer into 1.5 mL tube. The final eluate was centrifuged at 500×g for 5 min at 4° C. The pellet of isolated dendritic cells was resuspended in 1 mL TRIzol reagent and then frozen at −80° C. Flow cytometric analysis of aliquots of the isolated cells showed presence of at least 94% of CD11c-positive cells in total cell population.

Reverse Transcription and qPCR.

RNA (250-1000 ng) isolated from total splenocytes and DCs was reverse transcribed using GoScript Reverse Transcription System (Promega; cat #A5001) exactly as described in the manufacturer's protocol. Aliquots (1 μL) from the reverse transcribed samples were PCR amplified on Stratagene MX3005P using RT2 SYBR Green ROX qPCR Mastermix (QIAGEN; cat #330522) and primers for beta-actin (QIAGEN; cat #PPM02945A-200), Indoleamine 2,3-dioxygenase (forward primer: 5′-TGGCGTATGTGTGGAACCG-3′ [SEQ ID NO:3] and reverse primer: 5′-CTCGCAGTAGGGAACAGCAA-3′ [SEQ ID NO:4]) or CD11c (forward primer: 5′-CTGGATAGCCTTTCTTCTGCTG-3′ [SEQ ID NO:5] and reverse primer: 5′-GCACACTGTGTCCGAAC TCA-3′ [SEQ ID NO:6]) genes. Relative gene expression levels were determined by the 2(-Delta Delta C(T)) (“2-ΔΔCt”) method (Livak and Schmittgen, 2001 Methods; 25(4):402-8.) by normalizing the average ΔCt values against the average ΔCt values of beta-actin as an endogenous reference gene. Standard errors of mean were calculated for each group.

Results.

As shown in FIG. 16, statistically significant increases in IDO mRNA levels were detected in splenic DCs from NOD mice treated with KLK1. Specifically, an increase in IDO mRNA was noted in DCs isolated from animals receiving the medium and high doses of KLK1, and the high dose of KLK1 every 3 days. Little or no increase in IDO mRNA was detected in the DC's of mice receiving low doses of KLK1 and only a moderate increase in IDO mRNA was detected in DC's from animals receiving KLK1 once a week.

Increased IDO mRNA levels in the splenic dendritic cells correlated with the level of attenuation of the autoimmune response observed in the various KLK1 treatment groups (supra). Specifically, statistically significant increases in IDO were observed in splenocytes from low, medium and high daily doses of KLK1 treated animals compared to negative control group, with highest IDO levels detected in animals treated with the highest KLK1 dose. Similarly, in DC's, significantly higher IDO levels were detected in DC's from animals treated with medium and high, and high 3× week, and high 1× week. These results suggest KLK1 treatment may result in higher increases in IDO expression in DC's compared to splenocytes. Increased dose and increased dose frequency of KLK1 resulted in increased Treg and decrease in the incidence of development of diabetes, and increased IDO mRNA levels.

### Example 5

Reversal of Resent Onset T1D with KLK1.

In this experiment, KLK1 is administered to determine if treatment can reverse early onset diabetes in NOD mice, analogous to human T1D patients in the Honeymoon Phase. Once the NOD mice become diabetic the majority of beta cells are destroyed by the autoimmune reaction, but some beta cells remain. If the autoimmune reaction can be attenuated, the remaining beta cells can be protected from autoimmune insult and thus replicate to repopulate the islets to some extent and/or produce increased levels of insulin.

Female mice NOD/ShiLtJ are purchased at 4 weeks of age. The mice are provided food and water provided ad libitum. The onset of spontaneous diabetes is identified by assessing urine glucose levels with Diastix strips (Bayer, Tarrytown, N.Y.) and verified by blood glucose measurement using an Ascensia Elite 1 one-touch blood glucose monitor (Bayer). Mice with blood glucose levels >250 mg/dl for three consecutive measurements are considered diabetic. Porcine insulin (Sigma; ≧27 USP units/mg; 15-20 units/kg; one injection every 2-3 days) is injected subcutaneously into female NOD mice that had already developed acute spontaneous diabetes. Serum is obtained from non-fasting NOD with recent onset T1D. At least 5 animals are sacrificed and various tissues and cells isolated, including pancreas for histological assessment of beta cell mass, insulitis, ratio of CD4+/CD8+ cells in islet infiltrates; spleen to determine the percent lymphocytes that are CD8+, IDO mRNA levels in splenocytes and dendritic cells (DCs); the percent of CD4+ that are CD25+/Foxp3+; and the percent lymphocytes in PLN that are CD8+.

After diabetes is established in a mouse, it is randomly placed in one of 4 groups: Group 1, negative control, vehicle only “vehicle”); Group 2, 0.08 Units KLK1 daily (“low daily”); Group 3, 0.4 Units KLK1 daily (“medium daily”); and Group 4, 2 Units KLK1 daily (“high daily”). From 5 to 8 mice per group may be investigated. The vehicle or KLK1 is administered via intraperitoneal injection. Insulin injections and KLK1 treatment are continued for 30 days. Insulin injections are stopped at least 3 days prior to the end of the experiment. Animals are sacrificed and serum, cells and tissues are isolated for analysis.

The KLK1 treatment would attenuate the autoimmune reaction attacking the beta cells. This would be evident in a dose dependent decrease in insulitis after about 30 days of KLK1 treatment compared to newly diabetic animals. The ratio of CD4+/CD8+ cells in islet infiltrates would increase in a dose dependent manner after about 30 days of KLK1 treatment compared to newly diabetic animals, indicative of reduced insulitis. In isolated spleen there is an increase in the percent lymphocytes that are CD8+, and an increase in the percent of CD4+ that are CD25+/Foxp3+ after about 30 days of KLK1 treatment compared to newly diabetic animals. The percent lymphocytes in PLN that are CD8+ decrease after about 30 days of KLK1 treatment, in a KLK1 dose dependent manner. Additionally, IDO mRNA levels in splenocytes and DC's increase after about 30 days of KLK1 treatment animals compared to newly diabetic animals. In Group 1 animals treated with vehicle (negative control), the above parameters are expected to be relatively unchanged or worsen compared to newly diabetic NOD mice.

Attenuation of the autoimmune reaction that resulted in T1D in NOD mice may allow remaining beta cells to replicate and replenish some of the beta cells. This repopulation of the beta cells may be detected by an increase in beta cell mass in animals treated with KLK1 for 30 days compared to newly diabetic NOD mice. NOD mice treated with vehicle are not expected to have an increase in beta cell mass and may have the same levels or worsen as compared to newly diabetic animals. One outcome may be in C-peptide and insulin levels, where animals newly diagnosed with T1D would have very low to no detectable insulin or C-peptide, and after 30 days of KLK1 treatment, the NOD mice would have some detectable level of C-peptide. Detecting such an increase would suggest some beta cells have replicated and are producing and secreting insulin. The most dramatic would be restoration of a proper response to glucose in an IPTGG in the 30 day KLK1 treatment group. In NOD animals after spontaneously developing T1D and 30 days of high dose KLK1 treatment, a IPGTT response similar to that seen in FIG. 4B would indicate the beta cells have regenerated to the point that they can respond to a glucose challenge.

### Example 6

Reversal of Established Type 1 Diabetes in NOD Mice by Administering KLK1.

In this experiment, KLK1 is administered to NOD mice 20 days after development of spontaneous T1D to determine if treatment can reverse established diabetes in NOD mice, analogous to human T1D patients with established diabetes. In previous studies, NOD with established T1D for 20 days, the majority of beta cells are destroyed by the autoimmune reaction, but some beta cells may remain in a quiescent state and no longer produce insulin. If the autoimmune reaction can be attenuated, the quiescent beta cells can rejuvenate, replicate and repopulate the islets, or the remaining cells may restart producing insulin to some extent in absence of the autoimmune attack.

Female mice NOD/ShiLtJ are purchased at 4 weeks of age. The mice are provided food and water provided ad libitum. The onset of spontaneous diabetes is identified by assessing urine glucose levels and verified by blood glucose measurement. Mice with blood glucose levels >250 mg/dl for three consecutive measurements are considered diabetic. Porcine insulin (Sigma; ≧27 USP units/mg; 15-20 units/kg; one injection every 2-3 days) is injected subcutaneously into female NOD mice that had already developed acute spontaneous diabetes. Insulin injections last for 20 days to ensure total destruction of any residual degranulated beta cells (analogous to established T1D in human patients described herein).

Serum is obtained from non-fasting NOD mice 20 days after the onset T1D. At least 5 animals are sacrificed and various tissues and cells isolated, including pancreas for histological assessment of beta cell mass, insulitis, ratio of CD4+/CD8+ cells in islet infiltrates, spleen to determine the percent lymphocytes that are CD8+, IDO mRNA levels in spleenocytes and dendritic cells (DCs), the percent of CD4+ that are CD25+/Foxp3+, and the percent lymphocytes in PLN that are CD8+.

After diabetes is established for 20 days, the mice are randomly placed in one of 4 groups: Group 1, negative control, vehicle only “vehicle”); Group 2, 0.08 Units KLK1 daily (“low daily”); Group 3, 0.4 Units KLK1 daily (“medium daily”); and Group 4, 2 Units KLK1 daily (“high daily”). From 5 to 8 mice per group may be investigated. The vehicle or KLK1 is administered via intraperitoneal injection. Insulin injections and KLK1 treatment are continued for 30 days. Insulin injections are stopped at least 3 days prior to the end of the experiment. Animals are sacrificed and serum, cells and tissues are isolated for analysis.

The KLK1 treatment would attenuate the autoimmune reaction attacking the beta cells. This would be evident a dose dependent decrease in insulitis after about 30 days of KLK1 treatment compared to 20 day “established” diabetic animals. The ratio of CD4+/CD8+ cells in islet infiltrates would increase in animals after 30 days of KLK1 treatment compared to established diabetic animals in a KLK1 dose dependent manner. In spleen there is an expected increase in the percent pf lymphocytes that are CD8+, and an increase in the percent of CD4+ that are CD25+/Foxp3+ after 30 days of KLK1 treatment animals compared to established diabetic animals. The percent lymphocytes in PLN that are CD8+ would be expected to decrease after 30 days of KLK1 treatment, in a KLK1 dose dependent manner. Additionally, IDO mRNA levels in splenocytes and DC's are expected to increase after 30 days of KK1 treatment animals compared to established diabetic animals. In Group 1 animals treated with vehicle (negative control), the above parameters are expected to be relatively unchanged or worsen compared to established diabetic NOD mice.

Attenuation of the autoimmune reaction that resulted in T1D in NOD mice may allow remaining beta cells to replicate and replenish some of the beta cells and/or restart insulin production. This repopulation of the beta cells may be detected by an increase in beta cell mass in animals treated with KLK1 for 30 days compared to established diabetic NOD mice. NOD mice treated with vehicle are not expected to have an increase in beta cell mass and may have the same levels or worse as detected in established diabetic animals. One outcome may be in C-peptide and insulin levels, where animals with established T1D would have very low to no detectable insulin or C-peptide, and after 30 days of KLK1 treatment, the NOD mice would have some detectable C-peptide. Detecting such an increase would suggest some beta cells have replicated and are producing and secreting insulin or remaining beta cells are sufficiently protected and able to produce insulin. The most dramatic would be restoration of a proper response to glucose in an IPTGG in the 30 day KLK1 treatment group. In NOD animals with established T1D and then treated about 30 days with high dose KLK1 treatment, an IPGTT response similar to that seen in FIG. 4B would indicate the beta cells have regenerated to the point that they can respond to a glucose challenge.

All publications, patent applications, and issued patents cited in this specification are herein incorporated by reference as if each individual publication, patent application, or issued patent were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

1. A method for treating a patient with type 1 diabetes (T1D) comprising administering a therapeutically effective amount of a KLK1 polypeptide to the patient, where the KLK1 polypeptide comprises an amino acid sequence at least 95% identical to residues 25-262 of SEQ ID NO:2, and where the KLK1 polypeptide retains an E145 substitution, an A188 substitution, or both, relative to SEQ ID NO:1.

2-4. (canceled)

5. The method of claim 1, where the KLK1 polypeptide retains an E145Q substitution, an A188V substitution, or both, relative to SEQ ID NO:1.

6. The method of claim 5, where the KLK1 polypeptide comprises residues 25-262 of SEQ ID NO:2.

7. The method of claim 1, where the patient is in the honeymoon phase or recent onset of T1D.

8. The method of claim 7, where the patient in the honeymoon phase or recent onset of T1D has about 10-20%) of their pancreatic beta cells relative to a healthy control or other reference standard, and produces insulin.

9. The method of claim 1, where the patient has established T1D.

10. The method of claim 1, where the patient has latent autoimmune diabetes of adults (LADA).

11. The method of claim 1, wherein the therapeutically effective amount of the KLK1 polypeptide attenuates an autoimmune reaction against the pancreatic beta cells in the patient.

12. The method of claim 1, further comprising the step of measuring the level of one or more biomarkers in the patient to assess the effectiveness of administering the KLK1 polypeptide, where the one or more biomarkers are selected from:
(a) C-peptide, where the serum C-peptide levels are increased compared to the serum C-peptide levels of the patient prior to onset of treatment with the KLK1;
(b) regulatory T cells (Tregs), where the umber or level of Tregs is increased compared to number or levels of Tregs in the patient prior to onset of treatment with KLK1;
(c) indoleamine-pyrrole 2,3-dioxygenase (IDO) expression, where the level of IDO expression is increased compared to levels in the patient prior to onset of treatment with KLK1;
(d) fasting blood glucose, where the fasting blood glucose level of the patient is reduced compared to the fasting blood glucose level of the patient prior to onset of treatment with the KLK1;
(e) HBA1c, where the HBA1c levels of the patient is reduced compared to the HBA1c levels of the patient prior to onset of treatment with the KLK1; and
(f) ketone bodies, where the level of ketone bodies in the patient is reduced compared to the level of ketone bodies prior to onset of treatment with the KLK.

13-15. (canceled)

16. The method of claim 12, where the level of IDO expression is characterized by IDO mRNA levels in splenic dendritic cells (DCs).

17. The method of claim 12, comprising maintaining or reducing the dosage amount and/or frequency of the KLK1 polypeptide upon increase in one or more of said biomarkers, optionally where increase in said one or more biomarkers has been maintained for at least about 1, 2, 3, 4, 5, 6, or 7 weeks prior to reducing the dosage amount and/or frequency.

18-21. (canceled)

22. A method for delaying onset of Type 1 diabetes (T1D) in a patient, where the patient does not have T1D but is at increased risk for developing T1D, comprising administering a therapeutically effective amount of a KLK1 polypeptide to the patient.

23. The method of claim 22, where the patient has one or more biomarkers associated with increased risk for developing TID.

24. The method of claim 23, where the one or more biomarkers are selected from the group consisting of HLA-DQB1(IDDM1) alleles associated with TID, increased antibodies against insulin, increased antibodies against islets, increased antibodies against glutamic acid decarboxylase (GAD), increased antibodies against IA2 (ICA512), increased circulating T cells that react with beta cell antigens, increased insulitis, increased inflammation of the pancreas, increased ketone bodies, decreased suppressor (regulatory) T cells (Tregs) (CD4+ cells that are also CD25+/Foxp3+), increased HbA1c levels, decreased C-peptide levels, and decreased IDO (Indoleamine-pyrrole 2,3-dioxygenase) levels, relative to a healthy control or reference standard.

25. The method of claim 24, where the patient has HbA1c levels of about 5.7% to 6.4%.

26. A method of reducing a CD8+ autoimmune response in a patient comprising administering a KLK1 polypeptide to the patient.

27. The method of claim 1, further comprising determining circulating C-peptide levels in the patient optionally prior to administration and following administration of a KLK1 polypeptide, wherein administration of the KLK1 polypeptide is continued until an increase in circulating C-peptide levels is observed.

28. The method of claim of claim 1, further comprising determining indoleamine-pyrrole 2,3-dioxygenase (IDO) levels in the patient optionally prior to administration and following administration of a KLK1 polypeptide, wherein administration of the KLK1 polypeptide is continued until an increase in IDO levels is observed.

29. The method of claim 28, comprising measuring IDO mRNA levels in splenic dendritic cells (DCs).

30. A method for determining efficacy of administrating a KLK1 polypeptide to a patient with type 1 diabetes (TID), comprising measuring the circulating C-peptide levels optionally prior to administration and following administration of a KLK1 polypeptide, wherein administration of a KLK1 polypeptide is continued until an increase in circulating C-peptide levels is observed.

31. The method of 30, wherein the dosage levels and/or dosage frequency of KLK1 polypeptide administered to the patient is increased until an increase in C-peptide levels is observed.

32. A method for determining efficacy of administrating a KLK1 polypeptide to a patient with type 1 diabetes, comprising measuring indoleamine-pyrrole 2,3-dioxygenase (IDO) levels optionally prior to administration and following administration of a KLK1 polypeptide, wherein administration of the KLK1 polypeptide is continued until an increase in IDO levels is observed.

33. The method of claim 32, comprising measuring IDO mRNA levels in splenic dendritic cells (DCs).

34. The method claim 32, where the dosage levels and/or dosage frequency of KLK1 polypeptide administered to the patient is increased until an increase in IDO levels is observed.

35. The method of claim 1, wherein the KLK1 is administered by subcutaneous injection.

36. An isolated KLK1 polypeptide, comprising (a) the amino acid sequence of SEQ ID NO:2, (b) residues 19-262 of SEQ ID NO:2, (c) residues 25-262 of SEQ ID NO:2, or (d) a variant thereof having an amino acid sequence at least 95% identical to (a), (b), or (c), where the variant retains an E145 substitution, an A188 substitution, or both, relative to SEQ ID NO: 1.

37. The isolated KLK1 polypeptide of claim 36, wherein the variant retains an E145Q substitution, an A188V substitution, or both, relative to SEQ ID NO:1.

38. The isolated polypeptide of claim 37, comprising residues 25-262 of SEQ ID NO:2.

39. The isolated polypeptide of claim 36, further comprising a heterologous fusion partner.

40-41. (canceled)

42. A host cell, comprising a recombinant form of the polypeptide of claim 36.

43. The host cell of claim 42, where the host cell is 293 cell or a CHO cell.

44. A pharmaceutical composition, comprising the KLK1 polypeptide of claim 36 and a physiologically acceptable carrier.

45-46. (canceled)
